(19) United States
(12) Patent Application Publication
(10) Pub. No.: US 2024/0290428 A1
(43) Pub. Date: Aug. 29, 2024

(54) METHOD OF ASSAY DESIGN

(71) Applicant: IMPERIAL COLLEGE INNOVATIONS LIMITED, London (GB)

(72) Inventors: Jesus RODRIGUEZ MANZANO, London (GB); Ahmad MONIRI, London (GB); Luca MIGLIETTA, London (GB); Pantelis GEORGIOU, London (GB)

(21) Appl. No.: 18/568,660

(22) PCT Filed: Jun. 10, 2022
(86) PCT No.: PCT/EP2022/065895

§ 371 Date: Dec. 8, 2023

(30) Foreign Application Priority Data
Jun. 10, 2021 (GB) 2108339.9

(51) Int. Cl. G16B 25/20; G16B 40/10

(52) U.S. Cl. CPC G16B 25/20; G16B 40/10

(57) ABSTRACT

Disclosed herein are methods and systems for determining optimal primer sets for a multiplex assay, each of the optimal primer sets intended to amplify one or more targets. The method comprises obtaining amplification data from a plurality of preparatory assays. The amplification data describes at least the amplification of a first target of the one or more targets by a first primer set in a first preparatory assay, the amplification of the first target amplified by a second primer set in a second preparatory assay, the amplification of a second target of the one or more targets by the first primer set in a third preparatory assay, and the amplification of the second target by the second primer set in a fourth preparatory assay. The method further comprises determining a plurality of similarity metrics, each similarity metric being indicative of a degree of similarity between the amplification data produced by one of the plurality of preparatory assays compared to another one of the preparatory assays. The method further comprises determining, based on the plurality of similarity metrics, the optimal primer sets for the multiplex assay.

This disclosure relates to a method and system for determining optimal primer sets for an assay, and in particular to determining optimal primer sets for a multiplex assay.

### BACKGROUND

Multiplex assays provide a practical solution for the detection of nucleic acids in a single reaction, reducing the resources needed such as time, cost, amount of sample, and reagents. This is important in many areas such as medical diagnostics and microbiology research.

However, for high-level multiplexing (e.g. 100 targets), the selection of primers becomes intractable since the number of possible multiplex assays grows exponentially. For example, when there are 9 targets, and 5 potential primer sets for each target, the number of possible multiplex assays is: 59=1,953,125 combinations.

Typically, methods for designing multiplex assays are in-silico. They rely on bioinformatic data, for example the most efficient single-plex assays. However, this is not necessarily indicative of the best classification performance. There are a number of considerations when optimizing primer design for a multiplex assay, and present methods of primer selection typically require multiple rounds of primer re-design, careful consideration of the relative abundance of a target with respect to primer concentration, and primer-primer competition.

For example, there is no need for multiple rounds of primer redesign as there is no primer-primer competition, and no need to consider the relative abundance of a target with respect to primer concentration. By contrast, there are a number of considerations when optimizing primer design for a multiplex assay. This method is therefore more time and resource efficient.

The present invention seeks to address these and other disadvantages encountered in the prior art by providing an improved method and system for determining optimal primer sets for a multiplex assay.

### SUMMARY

An invention is set out in the independent claims. Optional features are set out in the dependent claims.

According to an aspect, there is provided a computer-implemented method for determining optimal primer sets for a multiplex assay, each of the optimal primer sets intended to amplify one or more targets. The method comprises obtaining amplification data from a plurality of preparatory assays. The amplification data describes at least: the amplification of a first target of the one or more targets by a first primer set in a first preparatory assay; the amplification of the first target amplified by a second primer set in a second preparatory assay; the amplification of a second target of the one or more targets by the first primer set in a third preparatory assay; and the amplification of the second target by the second primer set in a fourth preparatory assay. The method further comprises determining a plurality of similarity metrics, each similarity metric being indicative of a degree of similarity between the amplification data produced by one of the plurality of preparatory assays compared to another one of the preparatory of assay. It is then determined, based on the plurality of similarity metrics, the optimal primer sets for the multiplex assay.

A similarity metric may be determined for each possible pairing of the preparatory assays.

The method may further comprise determining a viability score for each of a plurality of trial multiplex assays, the trial multiplex assays comprising trial primer sets, and the viability score being based on similarity metrics associated with each of the trial primer sets. Determining the optimal primer sets may be based on the plurality of similarity metrics comprises selecting the optimal primer sets from among the trial primer sets based on the ranking of the viability scores.

Determining the optimal primer sets may further comprise constructing a similarity matrix of similarity metrics, the similarity matrix representing every combination of target and primer set used in the preparatory assays. Sub-matrices may then be constructed from the similarity matrix, wherein each sub-matrix is indicative of a trial multiplex assay comprising trial primer sets, and the sub-matrix values are the similarity metrics associated with the trial primer sets. Each trial multiplex assay may then be assigned a viability score based on the similarity scores within each submatrix.

Determining the optimal primer sets based on the plurality of similarity metrics may comprise selecting the optimal primer sets from among the trial primer sets based on the viability scores.

Prior to determining a viability score, constraints may be applied to each sub-matrix of preparatory assays.

Determining the plurality of similarity metrics may comprise computing a distance measure between the data distributions of the one of the plurality of preparatory assays and the another one of the plurality of preparatory assays.

The distance measure may be one of Euclidean distance, Mahalonbis distance, Pearson Correlation, or Wasserstein distance.

The distance measure may be a shift-invariant Euclidean distance measure.

Assigning the viability scores to each trial multiplex assay may be based on a sum of the distances between the sub-matrix values.

Assigning the viability scores to each trial multiplex assay may be based on a minimum distance between any two of sub-matrix values.

Assigning the viability scores to each trial multiplex assay may be based on the product of the sum of the distances and the minimum distance.

The amplification data may be at least one of: melting curve data; amplification curve data; fluorescence intensity data; or non-fluorescence data such as electrochemical, colorimetric or pH-based signal data.

The preparatory assays may be singleplex assays.

At least some of the plurality of preparatory assays may be low-level multiplex assays, and the multiplex assay is a higher-level multiplex assay.

The amplification data may describe the amplification of a plurality of different combinations of targets by a plurality of different primers or primer sets.

The multiplex assay may be intended to identify a plurality of identifiable targets, and the optimal primer sets are intended to enable amplification of each of those identifiable targets to produce real-time amplification data from which the amplification activity of each identifiable target can be distinguished from the amplification activity of every other identifiable target.

According to another aspect of the present disclosure, a computer readable medium is provided comprising computer executable instructions which, when performed by a processor, cause the processor to perform the method of any preceding claim.

According to another aspect of the present disclosure, a system is provided comprising one or more processors, and a computer-readable medium including one or more instructions that, when executed by one or more processors, cause the system to perform the method of any preceding claim.

### FIGURES

Specific embodiments are now described, by way of example only, with reference to the drawings, in which:

FIG. 1 depicts a diagnostic workflow.

FIG. 2a depicts a process for nucleic acid amplification.

FIG. 2b is a graph depicting the typical profile of a negative and positive real-time amplification reaction, and in particular shows the change in pH or fluorescence over time in a DNA amplification reaction.

FIG. 3 depicts an assay development workflow.

FIG. 4 depicts a data analysis workflow.

FIG. 5 depicts a method according to the present disclosure.

FIG. 6 depicts an experimental workflow from singleplex to multiplex.

FIG. 7 depicts Final Fluorescent Intensity (FFI) similarity measurement for a single multiplex.

FIG. 8 depicts Amplification Curve Analysis (ACA) similarity measurements for a single multiplex.

FIG. 9 depicts Melting Curve Analysis (MCA) similarity measurements for a single multiplex.

FIG. 10a depicts digital PCR data for FFI in singleplex. FIG. 10b depicts FFI similarity measurements for each singleplex.

FIG. 11a depicts digital PCR data for ACA in singleplex. FIG. 11b depicts ACA similarity measurements for each singleplex.

FIG. 12a depicts digital PCR data for MCA in singleplex. FIG. 12b depicts MCA similarity measurements for each singleplex.

FIG. 13a depicts a MinScore vs SumScore scatter plot for FFI data. FIG. 13b depicts the distribution of the figure of merit (MinScore multipled by SumScore) for FFI data. FIG. 13c shows experimental validation for FFI data.

FIG. 14a depicts a MinScore vs SumScore scatter plot for ACA data. FIG. 14b depicts the distribution of the figure of merit (MinScore multipled by SumScore) for ACA data. FIG. 14c shows experimental validation for ACA data.

FIG. 15a depicts a MinScore vs SumScore scatter plot for MCA data. FIG. 15b depicts the distribution of the figure of merit (MinScore multipled by SumScore) for MCA data. FIG. 15c shows experimental validation for MCA data.

FIG. 16a depicts a MinScore vs SumScore scatter plot for AMCA data. FIG. 16b depicts the distribution of the figure of merit (MinScore multipled by SumScore) for AMCA data. FIG. 16c shows experimental validation for AMCA data.

FIG. 17 depicts a case study of primers and targets.

FIG. 18 depicts the optimal multiplex assays determined from the similarity measurements.

FIG. 19 illustrates a block diagram of one implementation of a computing device.

### DETAILED DESCRIPTION

At the highest level, the present application relates to a method of optimising the design of a nucleic acid multiplex assay capable of identifying a plurality of targets. The method uses experimental data from preparatory assays, for example from preparatory singleplex assays, to perform this optimisation. In overview, the data acquired from each preparatory singleplex assay can be compared with the data acquired from every other preparatory singleplex assay to determine a similarity metric for each pairing of singleplex assays. The similarity metrics are indicative of a degree of similarity between the data from these assays, where this data is typically real-time amplification data. The optimal primer sets for the multiplex assays can then be determined, based on those similarity metrics.

Using this kind of data-driven method, which analyses all the possible combinations and ranks them based on a score, a manageable ‘optimal’ set of multiplex assays may be generated. The empirical data comes from singleplex experiments, which are inherently simpler and quicker to perform than multiplex assays since little optimization is required. According to methods of the present application, there is no need for multiple rounds of primer redesign as there is no primer-primer competition, and no need to consider the relative abundance of a target with respect to primer concentration. The present method of assay design is therefore more time and resource efficient.

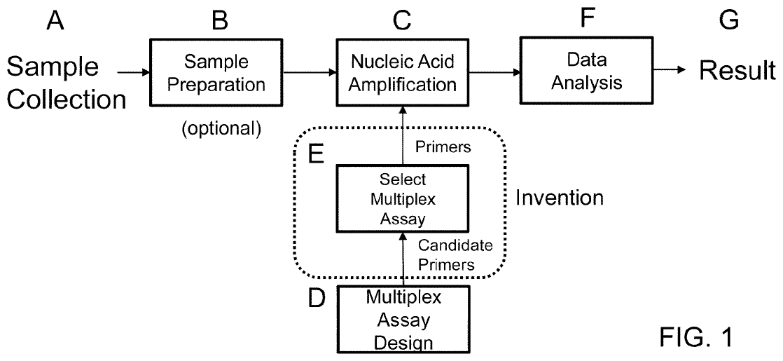

### FIG. 1

FIG. 1 depicts a high-level diagnostic workflow.

A. Sample collection may include, but is not limited to, clinical samples (from swabs, blood or tissue) and/or environmental samples (from water, soil or surfaces).

B. Sample preparation may include, but is not limited to sample enrichment, culturing and DNA/RNA extraction.

C. Nucleic Acid Amplification may include but is not limited to conventional qPCR or isothermal amplification (LAMP or RPA) in real-time bulk or single-molecule (i.e. digital PCR).

D. Multiplex Assay Design may include candidate primers being developed based on several factors such as primer length, GC content, melting temperature, primer cross-reactivity and primer dimer.

E. Select Multiplex Assay may include an ‘optimal’ multiplex assay being chosen based on data analysis performed on single-plex reactions in a manner which will be disclosed in more detail herein.

F. Data Analysis may include classification of the targets performed via methods such as final fluorescent intensity (FFI), melting curve analysis (MCA), amplification curve analysis (ACA), or amplification and melting curve analysis (AMCA).

G. The Result is the outcome of multiplexing (i.e. identification/diagnosis).

The present application discloses methods suitable for optimising step E. and in particular discloses a method of optimising the selection of primer sets required for a multiplex assay capable of producing the results required at step G.

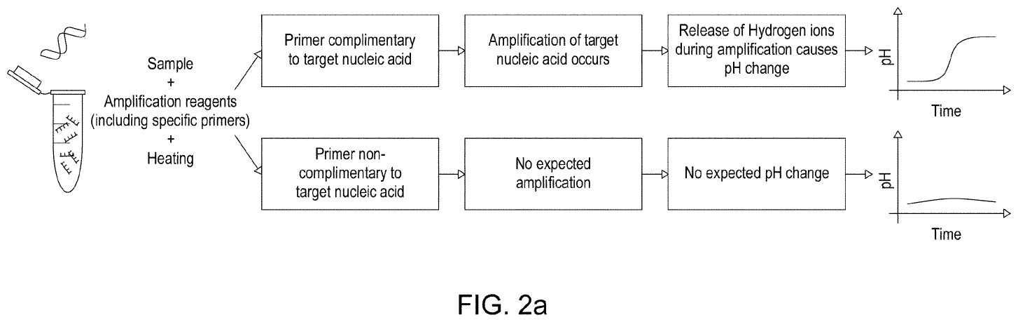

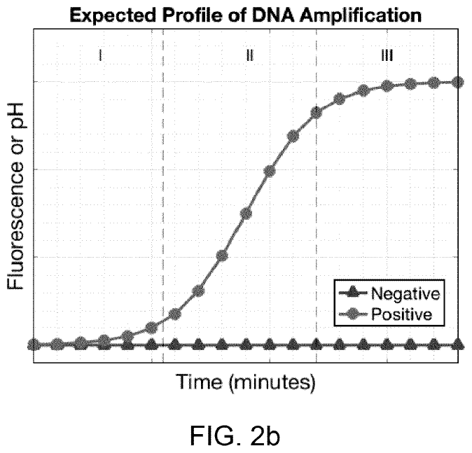

### FIG. 2

FIG. 2a depicts a process for nucleic acid amplification. FIG. 2b is a graph depicting the typical profile of a negative and positive real-time amplification reaction, and in particular shows the change in pH or fluorescence over time in a DNA amplification reaction

The following explanation of nucleic acid amplification relates primarily to pH based detection, and describes this detection primarily in relation to detecting DNA. This section serves to give useful background information and serves to give the reader an introduction to these concepts. However, the present disclosure is in no way limited to pH based detection, or to the detection of only DNA.

DNA amplification, the process of replicating DNA from one original DNA molecule, is used to amplify a single or a few copies of a segment of DNA generating thousands to millions of copies of a particular DNA sequence and can be used to determine whether a sample of human fluid or tissue contains DNA or RNA of a pathogen (such as viruses, bacteria, fungi or protozoa). The basic premise is that the DNA amplification is allowed if and only if the target pathogen exists. Following this, the DNA amplification is monitored. For instance, in traditional methods such as real-time polymerase chain reaction (PCR) each time a new amplicon is produced, a fluorescent molecule is released. Hence, the release of this fluorescent molecule is an indication of the presence of a pathogen in the sample.

It is also possible to monitor the pH of the chemical solution because during DNA amplification, each time a nucleotide is incorporated into the new DNA strand, Hydrogen ions are released which cause a change in the pH (pH=−log 10 [H+], where H+ is the concentration of Hydrogen ions or protons). The chemistry is summarised in the below equation where a is an integer constant.

DNA+reactants→2·DNA+α·Proton (H+)+products

If DNA amplification is triggered (i.e. the pathogen is present in the sample) then the reaction is defined as positive, otherwise, the reaction is described as negative.

A high-level description of how pH-based DNA detection is typically performed is illustrated in FIG. 2a and summarised in the following steps:
1. Chemical solution consisting of sample and other necessary chemicals is prepared.
2. Amplification reagents associated with a specific pathogen is added to the solution. This consists of a primer, a sequence of bases, that complements the target DNA.
3. Depending on the method of DNA detection, the chemical solution may be heated.
4. Amplification is triggered if the primer complements the DNA in the sample.
5. DNA amplification is monitored; for instance, through fluorescence or pH.

Assuming no noise exists in the system, a typical output profile for DNA detection is shown in FIG. 2b. This figure includes a typical profile for a positive and a negative reaction. The graph shows time on the x-axis, and pH (or fluorescence) on the y-axis. The graph is split into three ‘stages’ representing the expected profile for DNA amplification. At stage I) the reactants have not found each other yet. At stage II) amplification is taking place. At stage III) the reaction has saturated. The ‘time to positive’, tp, is defined as the time from the beginning of the reaction until a positive determination that the DNA is amplifying. Since the threshold is arbitrary, in examples used herein tp may be taken as the time for half of the amplification to complete.

Polymerase chain reaction (PCR), is the most common method of nucleic acid-based detection, within which the DNA amplification is done in cycles. In each cycle, the number of DNA molecules is doubled until one of the reactants have been consumed. Each PCR cycle typically comprise three steps (denaturation, annealing and extension) and each of these steps occur at a particular temperature. PCR has an appealing property that the number of DNA molecules can be easily quantified (2N, where N is the number of cycles).

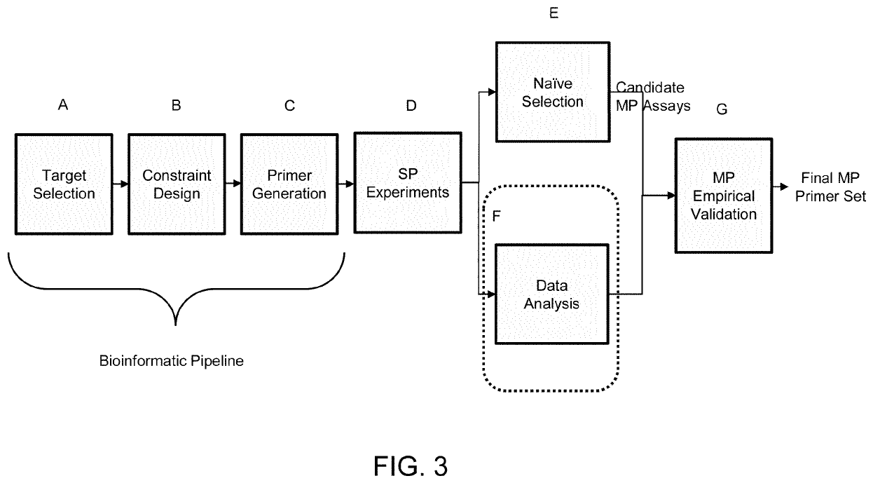

### FIG. 3—Assay Development Workflow

FIG. 3 depicts an assay development workflow. In prior methods, selection of a multiplex assay is a naïve selection, such as selecting the most efficient single-plex assays, which is not necessarily indicative of the best classification performance. In the present application, candidate multiplex assays are chosen systematically based on data from singleplex assays.

FIG. 3 shows both of these alternative options of generating candidate multiplex assays, via block E (“Naïve Selection” in accordance with the prior art) and step F (“Data Analysis”, according to methods and implementations of the present application).

A singleplex (SP) assay is used to amplify a single target in a single preparation. It may be used to detect one target sequence of DNA or RNA, to detect a specific virus or bacteria, or determine if an individual has a specific gene of interest.

A multiplex (MP) assay is used to detect two or more target sequences of DNA or RNA simultaneously, within a single sample preparation and amplification. Multiple sets of primers may be included to allow multiple targets to be detected within a single preparation.

Singleplex assays are inherently simpler since there is no need for multiple rounds of primer redesign as there is no primer-primer competition, and no need to consider the relative abundance of a target with respect to primer concentration. Singleplex assays are therefore quick and simple to perform, with little optimization required.

There are a number of considerations when optimizing primer design for a multiplex assay. For example, if one target is much more abundant than another, the primer concentration of the more abundant target may need to be limited to avoid it depleting reaction components for the lower abundance target.
A. Target selection. B. Constraint selection.
C. Primer selection.
D. Preparatory singleplex experiments.
E. Naïve selection of primers/primer sets, e.g. according to prior methods.
F. Alternative, data analysis stage, resulting in a determination of an optimal primer set according to methods disclosed herein.
G. Empirical validation of the multiplex assay designed according to either E or F.

Blocks A, B, and C are part of the bioinformatic pipeline and are three examples of selections that may be considered as part of primer set development.

At Block A (target selection), the panel to be developed is considered. For example, for respiratory tract infections, viruses such as flu A, flue B, COVID, RSV, etc. may be commonly targeted. Once the target is selected, a bioinformatics analysis is needed which involves going into a sequence database (such NCBI) and retrieve all the sequences available in the database for the selected targets.

One targets are selected at Block A, the primer design process takes place at Block B (constraint selection). To achieve a good assay design, there are a number of constraints on the primers. For example, melting temperature of the oligonucleotides, GC content, Hairpin formation, primer dimerization and prediction of melting curves.

After inputting your design constrains in the software (such as primer3 or bio python), primer sets will be generated and used for the first single plex screening (primer set).

At Block D (preparatory singleplex experiments), each single primer set is tested in diagnostic instrument (such qPCR).

In some embodiments, the preparatory assays may be low-level multiplex assays, which are used in order to optimize primer design for a high-level multiplex assay. For example, block D may be concerned with preparatory duplex or triplex assays. This may be a beneficial approach when the low-level multiplex assays are targeting the same gene or pathogen.

Block E (naïve selection) is part of routine multiplex development or assay design selection. It is common to try adding primer sets one by one and test the performance in the lab. This step is time and resource consuming, and not efficient when develop complex or high-level multiplex. If you have thousands of combinations, in order to select the best one all of the combinations must be manually tested in the lab which is inefficient.

Block F is an alternative to Block E which does not involve lab testing for all of the possible multiplex combinations. Instead, the methods set out in the present application provide a more efficient way for primer set selection which involves computing amplification data parameters for all the multiplex combinations using the similarity matrices.

At Block G (Empirical validation), validation of the top rank multiplex can be conducted both bioinformatically and in the wet lab. This step can be performed to evaluate that what the similarity measures outputted is true.

The final multiplex can then be selected.

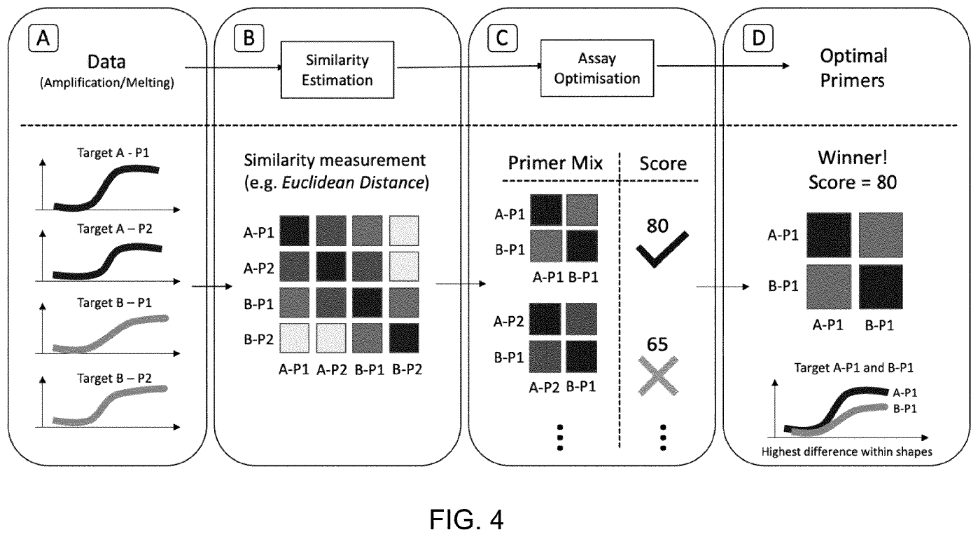

### FIG. 4

FIG. 4 depicts a workflow according to the present disclosure, using a simple example 2-plex problem (target A and target B).

At block A, amplification data is obtained for singleplex assay outputs across each of the two targets. For example, singleplex reactions in which the target A is amplified by primer 1 (Target A-P1), target A is amplified by primer 2 (Target A-P2), target B is amplified by primer 1 (Target B-P1), and target B is amplified by primer 2 (Target B-P2). These reactions may be described as preparatory reactions, because obtaining the real-time amplification data from these reactions serves as preparation for the task of optimising a multiplex assay design. The amplification data may be fluorescence data as used, for example, in Final Fluorescence Intensity (FFI) techniques; amplification curve data as used, for example, in Amplification Curve Analysis (ACA); melting curve data as used, for example, in Melting Curve Analysis (MCA); or both amplification curve and melting curve data as used, for example, in Amplification and Melting Curve Analysis (AMCA). The amplification data may also be non-fluorescence readout such as electrochemical, colorimetric and pH-based signals.

The amplification data may be real-time amplification data which can be described as amplification data collected over a time period. It may, for example, take the form of a time series. The real-time amplification data is indicative of a degree of amplification of a particular target, e.g. a particular nucleic acid, over time. The amplification data may alternatively be an end point measure. The amplification data obtained from each SP assay may be stored on a computer storage medium for later retrieval. This example uses amplification data for singleplex assays, however this method could also be applied to multiplex assays. For example, low-level multiplex assays (such as duplex or triplex assays) can be used in order to optimize primer design for a high-level multiplex assay.

At block B, similarity measurements are obtained for each combination of primer sets, or, optionally, for each viable combination of primer sets. For example, it may be redundant to compute the similarity between two primer sets for the same target and so the viable combinations are ones where there are different targets. Obtaining similarity measurements may comprise determining a similarity metric. The similarity metrics describe how similar the amplification data obtained from one SP assay is to the amplification curve data obtained from a second SP assay. For example, a similarity metric may be indicative of how similar the data obtained from a first assay, in which a target A is amplified by primer P1, is to data obtained from a second assay, in which a target B amplified by primer P2. In this way, the similarity between every single-plex experiment is computed. These similarity metric values can be set out in a similarity matrix, as shown schematically in block B. Determining the plurality of similarity metrics shown in block B may comprise computing a distance measure between the data distributions of the data obtained at block A.

The similarity metrics may be computed using a distance measure such as: Euclidean distance. Mahalonobis distance, Pearson Correlation, Wasserstein distance, or a shift invariant Euclidean distance. Finding the Euclidean distance between two amplification curves of 45 point time-series may involve considering each of the curves as a point in 45 dimensional space. The Euclidean distance can then be calculated between two points representing two amplification curves. If there are two data sets, an ‘aggregated’ Euclidean distance may be created. This may be achieved by averaging the curves from both data sets and computing the distance between the averages. It may also be achieved by computing many distances and then averaging afterwards. Shift invariant Euclidean distance may be implemented by shifting one of the curves from left to right (for example) and taking the minimum Euclidean distance. Another way this distance measure may be implemented is to align (for example) the middle point of the amplification curves and then compute that distance.

At block C, sub-matrices of primer sets and targets are constructed. Each sub-matrix is assigned a score based on the similarity metrics obtained at block B, for example using a predefined metric which uses the similarity metrics as an input. The score may be described as a multiplex “success score” and/or a “viability score”, and is indicative of how “distinguishable” the targets would be in a multiplex assay using the primer sets associated with that sub-matrix. A sub-matrix is indicative of a multiplex assay design. Block C depicts a first sub-matrix comprising a first trial primer mix, Target A-P1 and Target B-P1, and a second sub-matrix comprising a second trial primer mix, Target A-P2 and Target B-P1, but in a preferred implementation every possible sub-matrix of this form is constructed.

The predefined metric used to generate the multiplex success/viability score may be the sum of the similarity metrics of all the targets (“SumScore”). Optimising based on this predefined metric will optimize the overall distance between all the target data. For instance, when observing melting curves, the larger the SumScore, the more spread out the amplification melting curves are from each other. The predefined metric may be the minimum distance between any two targets (“MinScore”). Although optimizing this objective does not maximize the overall spread of the curves, it will ensure that the classification performance is good between any 2 targets. The predefined metric may also be a combined metric, for example a “Figure of Merit” obtained by multiplying the “SumScore” and the “MinScore”).

The sub-matrices produced at block C are indicative of trial multiplex assays comprising trial primer sets. The trial primer sets are taken from the plurality of primer sets tested at block A. The viability score determined for each trial multiplex assay is based on similarity metrics associated with each of the trial primer sets. For example, the SumScore or MinScore metrics may be used to determine the viability/success score. In an implementation, a sub-matrix is constructed for every possible target and primer set tested at block A. Once a viability score has been determined for each trial assay, i.e. when a viability score has been determined for each sub-matrix of targets and trial primer sets, the optimal primer set for the final multiplex design may be selected at block D from among the trial primer sets based on whichever trial multiplex assay has the best viability score.

In block D, N primer sets are output as optimal primers based on the ranking of the assigned scores as determined in block C. N is an arbitrary number which may be chosen based on the lab resources or the time or cost constraints on the project. These candidate assays may then be subsequently empirically validated in the lab in order to choose the final multiplex assay. The most successful and/or viable candidates for multiplex assays can be determined by comparing the success/viability scores determined at block C.

In general, for the optimisation of a multiplex assay capable of detecting N targets, and where M primer sets are to be tested as part of the assay design process, block A may comprise obtaining real-time amplification data from M×N singleplex assays. This may result in a similarity matrix at block B of size MN×MN. At block C, every possible unique sub-matrix of size N×N is assessed and a success/viability metric is obtained for each sub-matrix based on the similarity metrics determined at block B. However, in some cases each target may have a different number of primer sets to be tested. For example, a 3-plex assay may have M₁, M₂, and M₃ number of primer sets respectively. The output of block A would be (M₁+M₂+M₃)×N and the output of block B would be (M₁+M₂+M₃+ . . . +MN)N×(M₁+M₂+M₃)N.

The following is a brief summary of an implementation of the method shown in FIG. 4.

When it is desirable to design an optimised multiplex assay capable of identifying a plurality of identifiable targets, for example N identifiable targets, the method comprises obtaining real-time amplification data from preparatory assays involving those identifiable targets. This might involve actually performing those preparatory assays to obtain the data, retrieving already-obtained data from a library of data, or a combination of these approaches. When it is necessary to perform the experiments, then at block A, a plurality of primers and/or primer sets are used to amplify each of the identifiable targets to obtain real-time amplification data associated with each target and each primer/primer set. A similarity matrix of similarity metrics is constructed at block B, where the similarity matrix contains a similarity metric for the data associated with every combination of target and primer set used in the preparatory assays. For example, where the final multiplex assay design is intended to identify N targets, and where M primers or primer sets are tested in the preparatory assays at block A, the similarity matrix may have a size of MN×MN.

At block C, sub-matrices are constructed from the similarity matrix, wherein each sub-matrix is indicative of (e.g. describes and/or represents) a trial multiplex assay comprising trial primer sets, and the sub-matrix values are the similarity metrics associated with the trial primer sets. The trial primer sets are selected from among the primer sets tested at block A. A viability score is assigned to each trial multiplex assay based on the similarity scores within each submatrix. The viability score can be described as a score which reflects how different the similarity metrics within the sub-matrix are.

The more ‘different’ the similarity metrics are within a given sub-matrix, i.e. the less similar the underlying real-time amplification data associated with each primer/primer set is, the better. This is because it is more likely that those trial primer sets can be used in a final multiplex assay design which is capable of identifying each of the desired identifiable targets, while also ensuring a high degree of distinguishability between the amplification activity associated with each target. I.e., an optimal primer set should enable amplification of each of the identifiable targets to produce real-time amplification data from which the amplification activity of each identifiable target can be distinguished from the amplification activity of every other identifiable target.

Therefore, once a viability metric has been assigned to each sub-matrix, determining the optimal primer sets may simply comprise selecting the optimal primer sets from among the trial primer sets based on the viability scores. This may comprise simply outputting the sub-matrix which represents the trial multiplex assay with the best viability score.

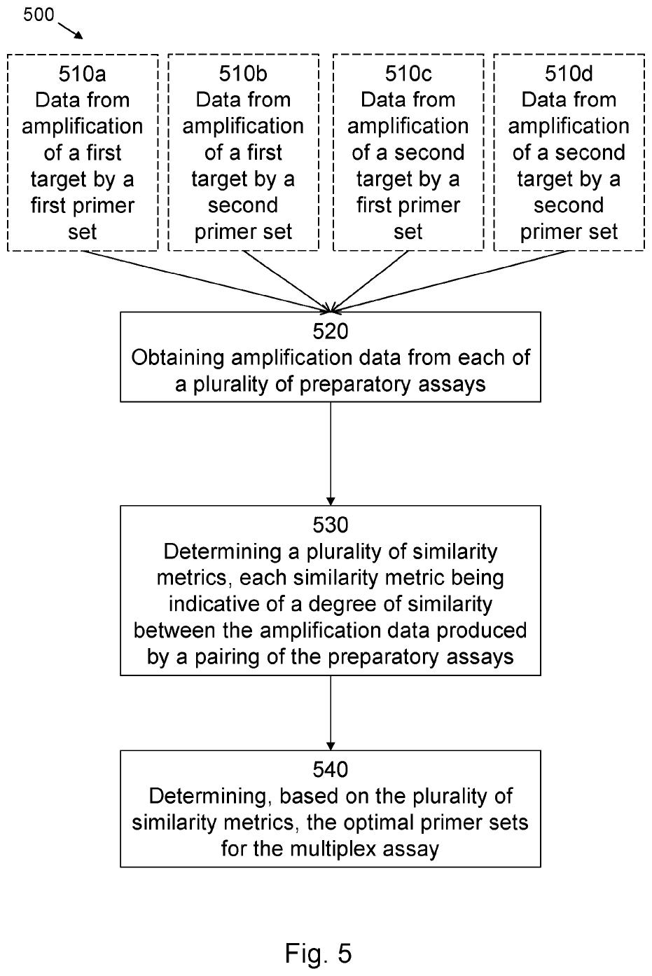

### FIG. 5

FIG. 5 is a flowchart depicting a computer-implemented method in accordance with the present disclosure. FIG. 5 acts as a summary of disclosed methods, for example the method depicted in FIG. 4 and described above. Dashed lines depict optional steps in the flowchart.

At blocks 510a, b, c, and d, data is obtained from a plurality of preparatory assays. These preparatory assays may be singleplex assays. Block 510a depicts obtaining amplification data from the amplification of a first target by a first primer, or primer set. Block 510b depicts obtaining amplification data from the amplification of a first target by a second primer, or primer set. Block 510c depicts obtaining amplification data from the amplification of a second target by a first primer, or primer set. Block 510d depicts obtaining amplification data from the amplification of a second target by a second primer, or primer set. The amplification data may be real-time amplification data, which can be described as amplification data collected over a time period.

Block 520 depicts obtaining amplification data from each of the plurality of preparatory assays (i.e., the data from blocks 510a, b, c, and d). In an implementation, this step may comprise retrieving the data associated with these preparatory assays from computer storage.

Block 530 depicts determining a plurality of similarity metrics, each similarity metric being indicative of a degree of similarity between the amplification data produced by a pairing (combination) of the preparatory assays.

Block 540 depicts the step of determining, based on the plurality of similarity metrics, the optimal primer sets for the multiplex assay.

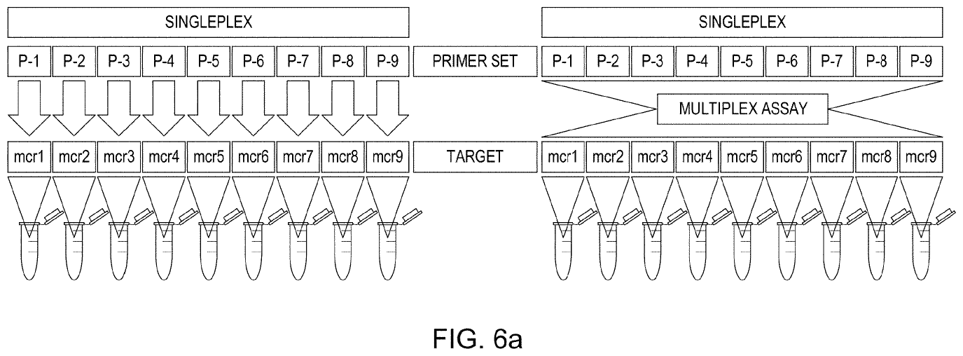

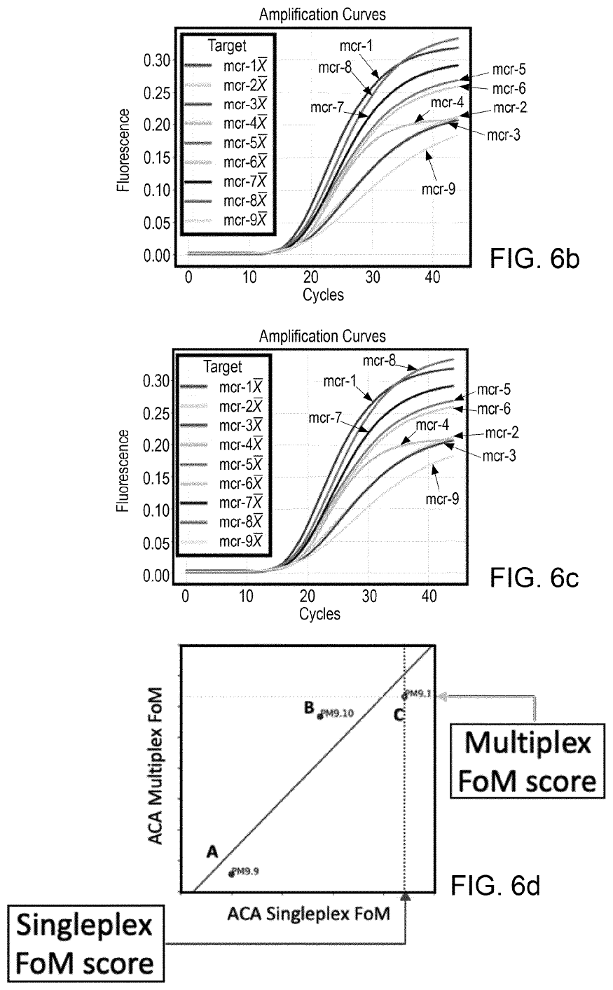

### FIG. 6—Experimental Workflow

FIG. 6a is a graph that depicts the difference within multiplex and singleplex assays. It illustrates singleplex assays for nine mcr targets (labelled mcr1 to mcr9) and 9 primer sets, as well as a multiplex assay for the same nine mcr targets and 9 primer sets. On the left, the figure shows how in a singleplex experiment each assay should have his own well dedicated; in the presence of the specific target this well will output an amplification signal. On the right, the figure shows how in a multiplex experiment each assay can be pooled in a single well; in the presence of any specific target this well will output an amplification signal.

FIGS. 6b and 6c are graphs that depicts Amplification Curves obtained from 9 sets of PCR primers for 9 different targets (mcr−1 to mcr−92), in singleplex and multiplex format respectively. FIG. 6b depicts the result when using singlplex assays, whereas FIG. 6c depicts amplification curves when using the same assays in a multiplex environment. The amplification of both is similar as same assays have been used, but the experimental setup is different (6b is singleplex and 6c is multiplex).

FIG. 6d is a graph that depicts the correlation within multiplex and singleplex Amplification Curve analysis (ACA) figure of merit (FoM). The X axis refers to ACA singleplex FoM and the Y axis refers to ACA multiplex FoM. As it can be seen, the linearity of the correlation indicates that the singleplex ranking (for each multiplex combination) from the similarity measures, is maintained when FoM is calculated in multiplex. FIG. 6d shows an example datapoint from a score determined from the singleplex Figure of Merit (FOM) against a score determined from the corresponding multiplex figure of merit. The Figure of Merit (FOM) score may be determined by multiplying together the “SumScore” and the “MinScore”. The linearity of the correlation demonstrates that there is experimental validation to show the association between the score from singleplex and multiplex lab experiments. The correlation between the singleplex and multiplex experiments means that knowledge can be translated between the two environments. In this case, the score is based on the Figure of Merit metric, although another predefined metric may also be used. Therefore, instead of trying 1,866,240 wet lab experiments (for a 9-plex assay with up to 6 primer sets for each target), only N primer sets need to be evaluated. N is an arbitrary number of optimal multiplex assays which are empirically validated in the laboratory. Project resources such as time and cost may impact the N which is selected.

### Types of Amplification Data

Examples of amplification data are fluorescence data, amplification curve data, and melting curve data. This data may be collected in real-time (in other words, collected over a time period) or as an end point measure.

Amplification curve data is indicative of an amplification reaction associated with at least one nucleic acid (target) present in the solution. The amplification curve data is indicative of the degree of amplification of target over time during the amplification reaction. Melting curve data is indicative of a degree of dissociation of a nucleic acid with increasing temperature.

Further examples of amplification data include non-fluorescence readout such as electrochemical, colorimetric and pH-based signals. Data may be generated from a variety of process/method, during or after the amplification event (i.e. electrophoresis and sequencing approaches).

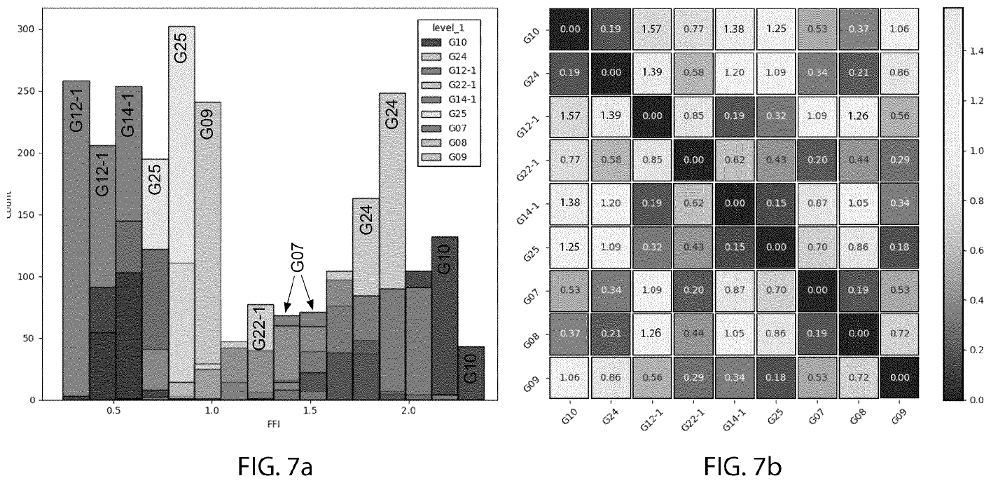

### FIGS. 7-18—Examples

FIG. 7a shows an example of final fluorescence intensity distributions. The Y axis represent the count of each assay, taking into account different replicates, and the X axis is the FFI value (from the amplification data or instrument read). As FFI can vary within small ranges the FFI for each primer set overlaps making difficult to visualise a clear distribution between different assay based only on FFI.

FIG. 7b shows an example of the similarity matrix based on Final Fluorescence Intensity (FFI) for 9 sets of primers for 9 different targets (one for each). Multiple replicates are used to construct a distribution of FFI values for each primer-target pair. The similarity metric used here is a distance measure, and in particular the distance measure used in this example is the Wasserstein distance.

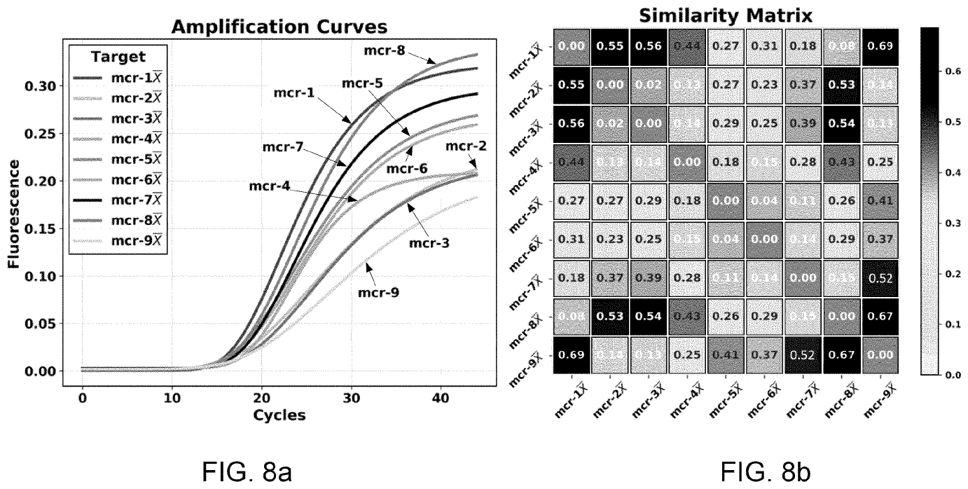

FIG. 8a is a graph that depicts the amplification curves obtained when using 9 sets of PCR primers in singleplex format for 9 different targets (mcr−1 to mcr−9). The axes indicated fluorescence values (X) and cycle numbers (Y). As can be seen, the amplification shape is different for each target. In FIG. 8b, the difference between the amplification shapes is computed using a shift-invariant Euclidean distance (used in this specific example as the similarity measure). The diagonal of the similarity matrix computes the distance within the same assay (or primer set) resulting in zero values as no difference is present. The rest of the confusion matrix shows the distance values for each assay compared to the other 8.

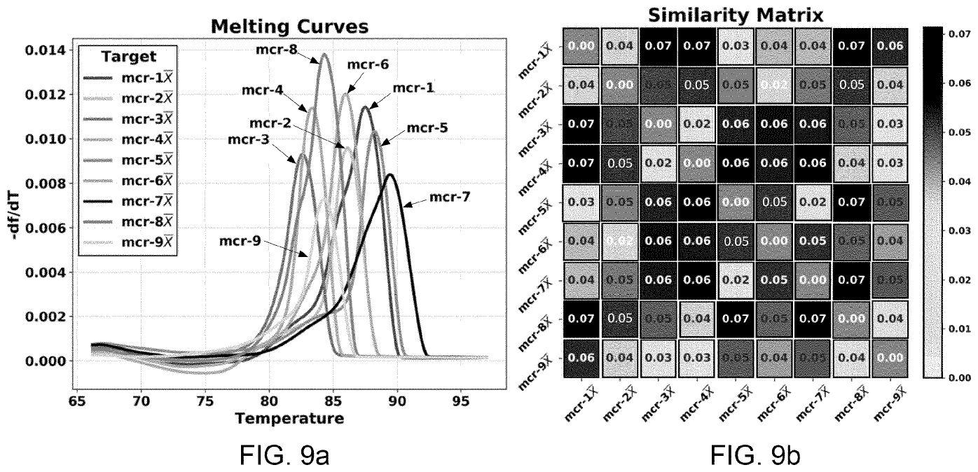

FIG. 9a is a graph that depicts the melting curves obtained when using 9 sets of PCR primers in singleplex format for 9 different targets (mcr−1 to mcr−9). The axes indicate the change in fluorescence level or −df/dT (X axis) and Temperature (Y axis). As it can be seen, the melting curves are different and specific for each mcr target, resulting in different peak height and distribution across temperatures. In FIG. 9b the difference between them is computed using Euclidean distance (used in this specific example as similarity measure). The diagonal of the similarity matrix computes the distance within the same assay (or primer set) resulting in zero values as no difference is present. The rest of the confusion matrix shows the distance values for each assay compared to the other 8.

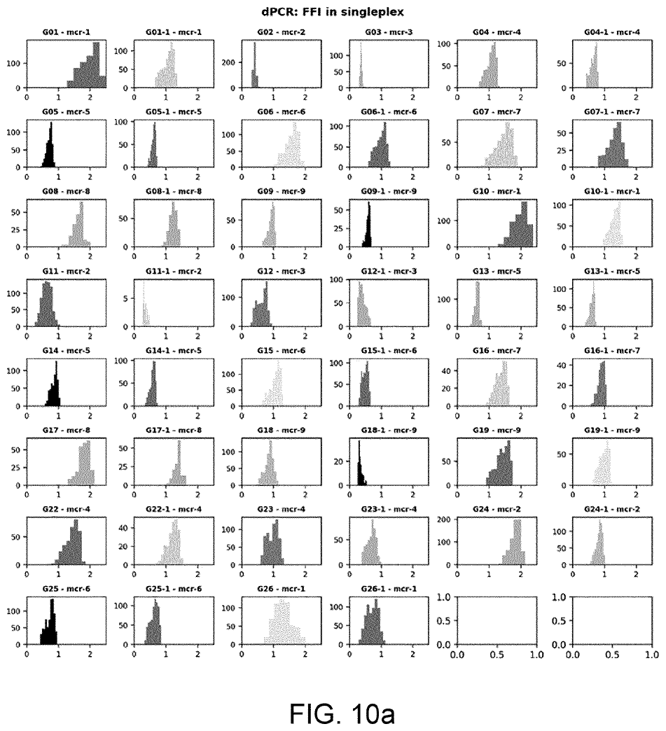

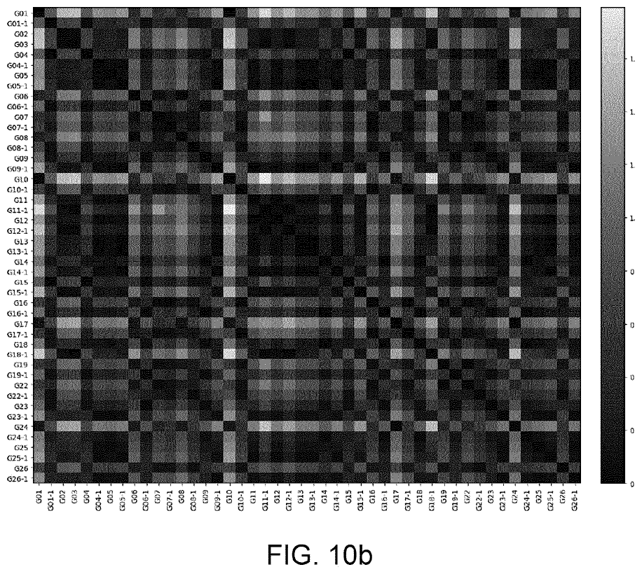

The examples shown in FIGS. 7a-b, 8a-b and 9a-b depict examples in which only a single primer set is used per target. However, multiple primer sets at different concentrations may be used. FIGS. 10-16a-b show more complex examples for a 9-plex assay detecting mobilised colistin resistant genes, with up to 6 primer sets for each target (in total 46 different single-plex experiments). The resulting 46×46 similarity matrix is therefore converted into 1,866,240 matrices which are 9×9 (each representing a potential multiplex). Subsequently, each 9×9 matrix is converted into a success or viability score and ranked from best to worse.

FIG. 10a is a graph that depicts the Final Fluoresence Intensity (FFI) distribution obtained across PCR replicates using 46 different singleplex assays. The Y axis of each subplot indicates the count (or distribution) for each FFI value obtained from each individual replicate and the X axis indicates the FFI value. FIG. 10b is a 46×46 similarity matrix (using Wasserstein distance) for all the singleplex. Both axes compare each singleplex with all the others. The diagonal of the similarity matrix computes the distance within the same assay (or primer set) resulting in zero values as no difference is present.

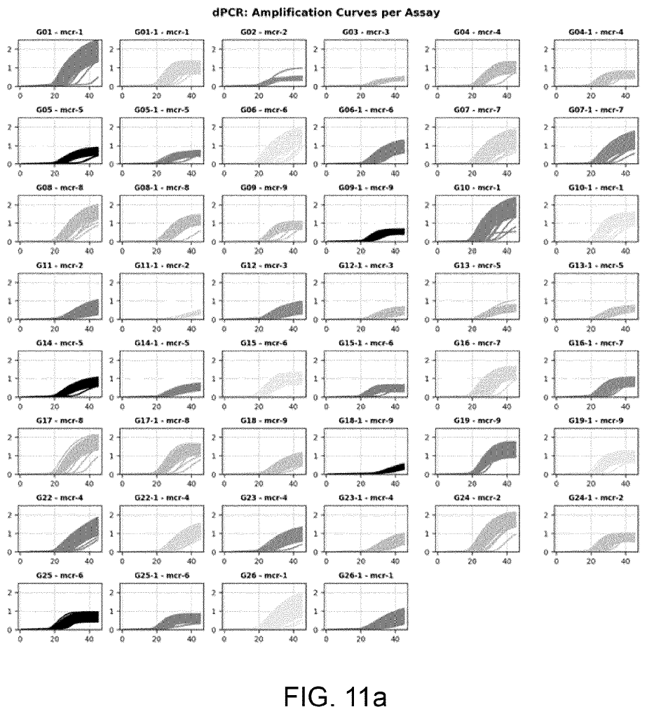

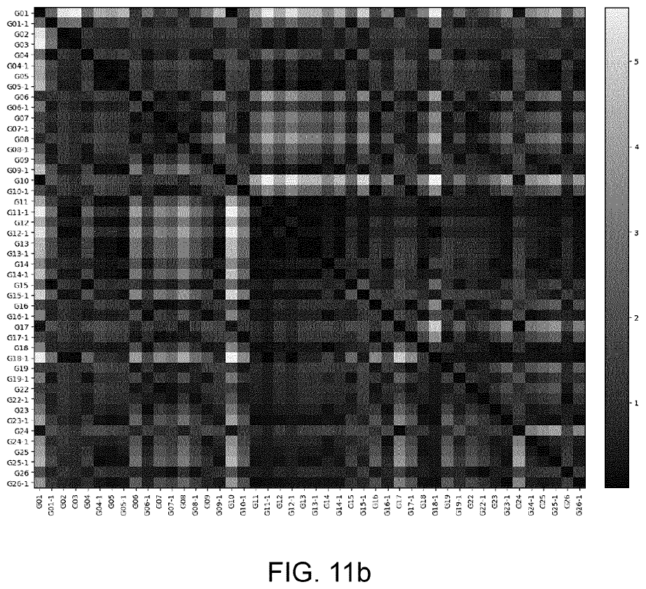

FIG. 11a is a graph that depicts the Amplification curves obtained across PCR replicates using 46 different singleplex assays. The axes indicate fluorescence values (X axis) and Cycle numbers (Y axis). The subsequent similarity matrix is generated based on a shift-invariant Euclidean distance. FIG. 11b is a 46 by 46 similarity matrix for all the singleplex tested in the wet lab. Both axes compare each singleplex with all the others. The diagonal of the similarity matrix computes the distance within the same assay (or primer set) resulting in zero values as no difference is present.

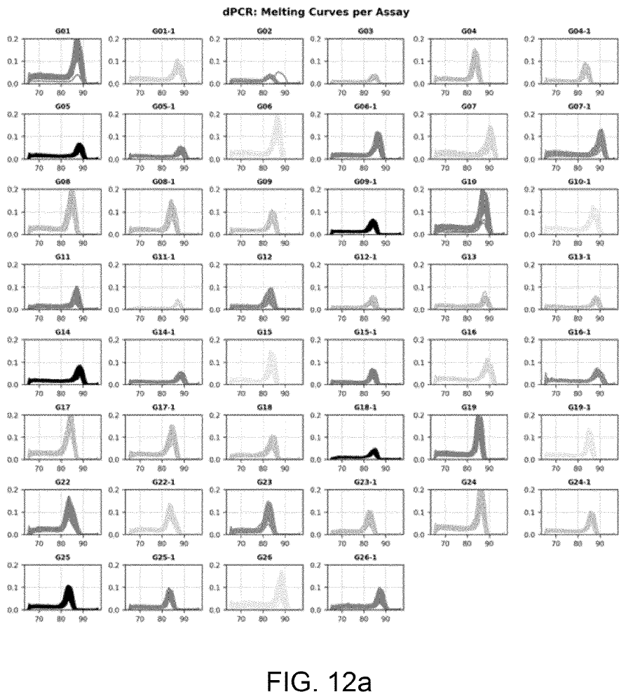

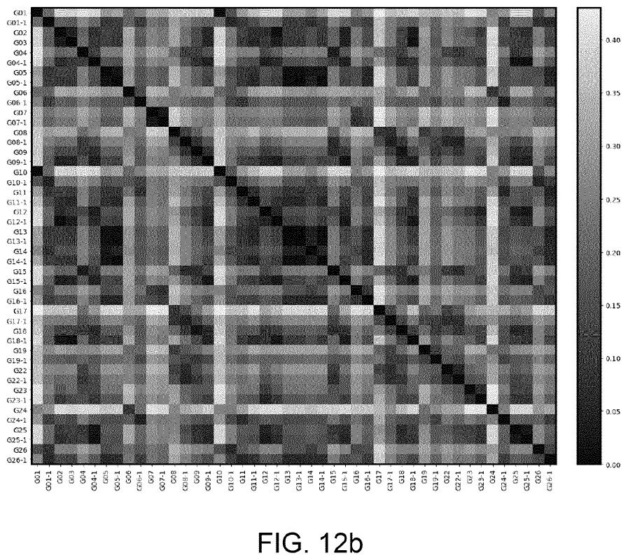

FIG. 12a is a graph that depicts the Melting curves obtained across PCR replicates using 46 different singleplex assays. The axes indicate the change in fluorescence level or −df/dT (X axis) and Temperature (Y axis). The subsequent similarity matrix is generated based on Euclidean distance. FIG. 12b is a 46 by 46 similarity matrix for all the singleplex tested in the wet lab. Both axes compare each singleplex with all the others. The diagonal of the similarity matrix computes the distance within the same assay (or primer set) resulting in zero values as no difference is present.

After the similarity matrices shown in FIGS. 10b, 11b and 12b are converted into the 1,866,240 9×9 matrices, each is subsequently assigned a success/viability score.

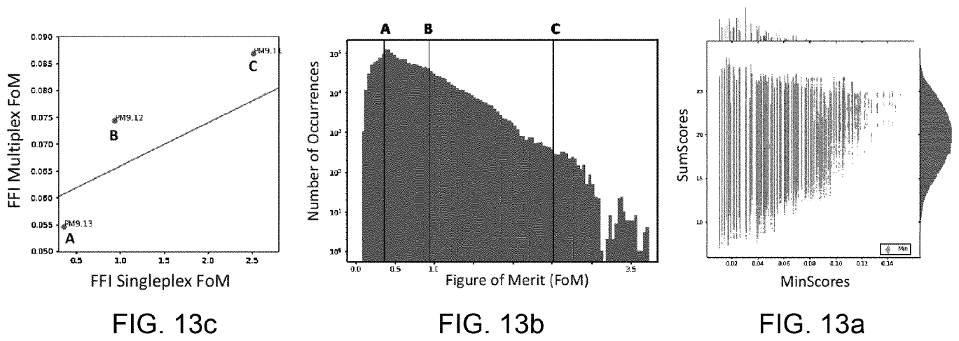

Each of FIGS. 13, 14, 15 and 16 contain the following graphs for FFI, MCA, ACA and AMCA respectively:
The left plot shows a MinScore vs SumScore scatter plot for all 1,866,240 combinations.
The middle plot shows the number of occurrences (i.e. distribution) of the figure of merit (i.e. MinScore×SumScore).
The right plot shows the experimental validation to show the association between the score from singleplex and multiplex lab experiments. If there is a correlation, then knowledge can be translated between the two environments. Therefore, instead of trying 1,866,240 wet lab experiments, only N primer sets need to be evaluated.

In FIG. 13, three graphs are shown depicting the correlation between singleplex and multiplex ranking system of the similarity measure for FFI values. A total of 1,866,240 combinations are computed and some of them may be tested in wet-lab to evaluate the correlation of the ranking system in the experimental setup. FIG. 13a depicts the distribution of all the possible combination based on SumScore (Y axis) and MinScore (X axis). Three selected assays are shown as case study. FIG. 13b depicts the distribution of all possible combination based on the computed figure of merit values (FoM). X axis represents the FoM value for each multiplex and the Y axis is the number of occurrences. The black line indicates where the selected assays are ranked. FIG. 13c depicts the correlation within both FoM values for the selected assays. The X axis represents the FoM values of singleplex assay and the Y axis the Multiplex FoM values. Both values refer to experimental data for the 3 selected assays, showing a linear correlation within both a multiplex and a singleplex setup.

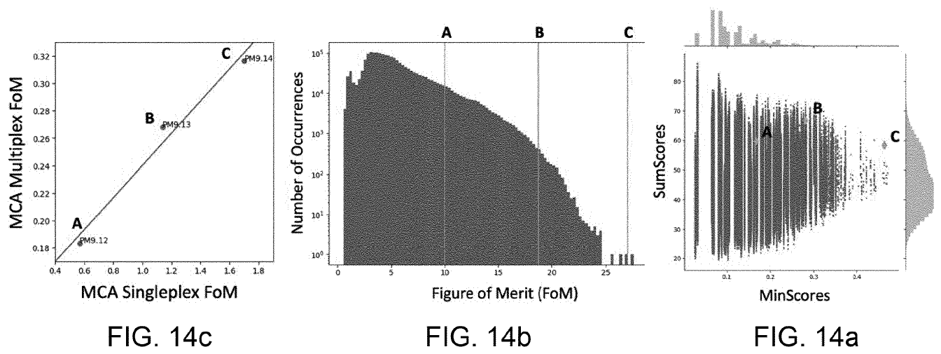

In FIG. 14, three graphs are shown depicting the correlation between singleplex and multiplex ranking system of the similarity measure for the ACA method. All the 1,866,240 combinations are computed and few of them tested in wet-lab to evaluate the correlation of the ranking system in the experimental setup. FIG. 14a depicts the distribution of all the possible combination based on SumScore (Y axis) and MinScore (X axis). 3 selected assays are shown as case study. FIG. 14b depicts the distribution of all possible combination based on the computed figure of merit values (FoM). X axis represents the FoM value for each multiplex and the Y axis is the number of occurrences. The black line indicates where the selected assays are ranked. FIG. 14c depicts the correlation within both FoM values for the selected assays. The X axis represents the FoM values of singleplex assay and the Y axis the Multiplex FoM values. Both values refer to experimental data for the 3 selected assays, showing a linear correlation within both a multiplex and a singleplex setup.

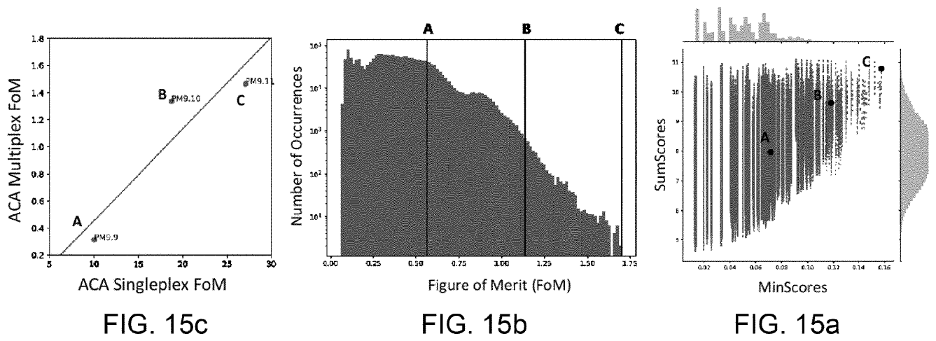

In FIG. 15, three graphs are shown depicting the correlation between singleplex and multiplex ranking system of the similarity measure for MCA method. All the 1,866,240 combinations are computed and few of them tested in wet-lab to evaluate the correlation of the ranking system in the experimental setup. FIG. 15a depicts the distribution of all the possible combination based on SumScore (y axis) and MinScore (x axis). Three selected assays are shown as case study. FIG. 15b depicts the distribution of all possible combination based on the computed figure of merit values (FoM). X axis represents the FoM value for each multiplex and the y axis is the number of occurrences. The black line indicates where the selected assays are ranked. FIG. 15c depicts the correlation within both FoM values for the selected assays. The x axis represents the FoM values of singleplex assay and the y axis the Multiplex FoM values. Both values refer to experimental data for the 3 selected assays, showing a linear correlation within both multiplex and singleplex setup.

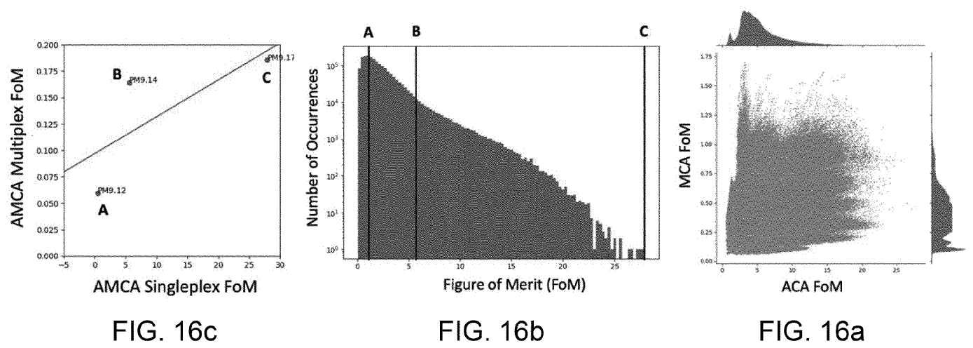

In FIG. 16, three graphs are shown depicting the correlation between singleplex and multiplex ranking system of the similarity measure for the AMCA method. All the 1,866,240 combinations are computed and few of them tested in wet-lab to evaluate the correlation of the ranking system in the experimental setup. FIG. 16a depicts the distribution of all the possible combination based on SumScore (y axis) and MinScore (x axis). Three selected assays are shown as case study. FIG. 16b depicts the distribution of all possible combination based on the computed figure of merit values (FoM). X axis represents the FoM value for each multiplex and the y axis is the number of occurrences. The black line indicates where the selected assays are ranked. FIG. 16c depicts the correlation within both FoM values for the selected assays. The x axis represents the FoM values of singleplex assay and the y axis the Multiplex FoM values. Both values refer to experimental data for the 3 selected assays, showing a linear correlation within both multiplex and singleplex setup.

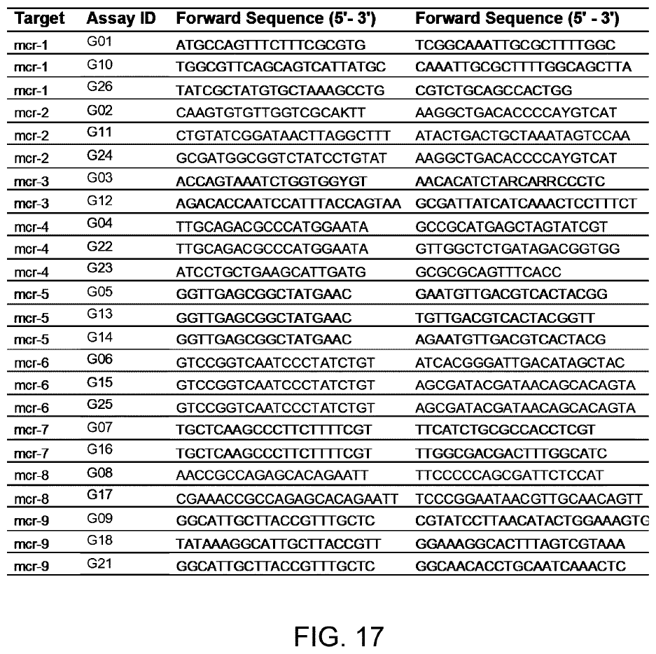

FIG. 17 shows the primer sequences and the generated candidate multiplex assays for the results in FIGS. 7 to 16. It includes the primer sequences and assay ID used.

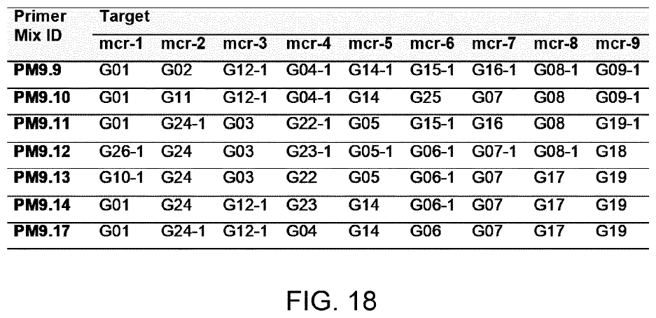

FIG. 18 shows the selected assays to demonstrate translation between single-plex and multiplex environments. By default, primer concentration is 500 nM and 250 nM for assays indicated by −1.

### The Biological Sample and Solution

The sample described at block A of FIG. 1 may be any suitable sample comprising one or more nucleic acids. For example, the sample may be an environmental sample or a clinical sample. The sample may also be a sample of synthetic DNA (such as gBlocks) or a sample of a plasmid. The plasmid may include a gene or gene fragment of interest.

The environmental sample may be a sample from air, water, animal matter, plant matter or a surface. An environmental sample from water may be salt water, waste water, brackish water or fresh water. For example, an environmental sample from salt water may be from an ocean, sea or salt marsh. An environmental sample from brackish water may be from an estuary. An environmental sample from fresh water may be from a natural source such as a puddle, pond, stream, river, lake. An environmental sample from fresh water may also be from a man-made source such as a water supply system, a storage tank, a canal or a reservoir. An environmental sample from animal matter may, for example, be from a dead animal or a biopsy of a live animal. An environmental sample from plant matter may, for example, be from a foodstock, a plant bulb or a plant seed. An environmental sample from a surface may be from an indoor or an outdoor surface. For example, the outdoor surface be soil or compost. The indoor surface may, for example, be from a hospital, such as an operating theatre or surgical equipment, or from a dwelling, such as a food preparation area, food preparation equipment or utensils. The environmental sample may contain or be suspected of containing a pathogen. Accordingly, the nucleic acid may be a nucleic acid from the pathogen.

The clinical sample may be a sample from a patient. The nucleic acid may be a nucleic acid from the patient. The clinical sample may be a sample from a bodily fluid. The clinical sample may be from blood, serum, lymph, urine, faeces, semen, sweat, tears, amniotic fluid, wound exudate or any other bodily fluid or secretion in a state of heath or disease. The clinical sample may be a sample of cells or a cellular sample. The clinical sample may comprise cells. The clinical sample may be a tissue sample. The clinical sample may be a biopsy.

The clinical sample may be from a tumour. The clinical sample may comprise cancer cells. Accordingly, the nucleic acid may be a nucleic acid from a cancer cell.

The sample may be obtained by any suitable method. Accordingly, the method of the invention may comprise a step of obtaining the sample. For example, the environmental air sample may be obtained by impingement in liquids, impaction on solid surfaces, sedimentation, filtration, centrifugation, electrostatic precipitation, or thermal precipitation. The water sample may be obtained by containment, by using pour plates, spread plates or membrane filtration. The surface sample may be obtained by a sample/rinse method, by direct immersion, by containment, or by replicate organism direct agar contact (RODAC).

The sample from a patient may contain or be suspected of containing a pathogen. Accordingly, the nucleic acid may be a nucleic acid from the pathogen. Alternatively, the nucleic acid may be a nucleic acid from the host.

The pathogen may be a eukaryote, a prokaryote or a virus. The pathogen may be found in or from an animal, a plant, a fungus, a protozoan, a chromist, a bacterium or an archaeum.

As used herein, “nucleic acid sequence” may refer to either a double stranded or to a single stranded nucleic acid molecule. The nucleic acid sequence may therefore alternatively be defined as a nucleic acid molecule. The nucleic acid molecule comprises two or more nucleotides. The nucleic acid sequence may be synthetic. The nucleic acid sequence may refer to a nucleic acid sequence that was present in the sample on collection. Alternatively, the nucleic acid sequence may be an amplified nucleic acid sequence or an intermediate in the amplification of a nucleic acid sequence.

As used herein, “anneal”, “annealing”, “hybridise” and “hybridising” refer to complementary sequences of single-stranded regions of a nucleic acid pairing via hydrogen bonds to form a double-stranded polynucleotide. As used herein, “anneal”, “anneals”, “hybridise” and “hybridises” may refer to an active step. Alternatively, as used herein, “anneal”, “anneals”, “hybridise” and “hybridises” may refer to a capacity to anneal or hybridise; for example, that a primer is configured to anneal or hybridise and/or that the primer is complementary to a target. Accordingly, for example, a reference to a primer or a region of a primer which anneals to a nucleic acid sequence or a region of a nucleic acid sequence may in a method of the invention mean either that the annealing is a required step of the method; that the primer or region of the primer is complementary to the nucleic acid sequence or region of the nucleic acid sequence; or that the primer or region of the primer is configured to anneal to the nucleic acid sequence or region of the nucleic acid sequence.

The term “primer” as used herein refers to a nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e. in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the nucleic acid primer typically contains 15 to 25 or more nucleotides, although it may contain fewer or more nucleotides. According to the present invention a nucleic acid primer typically contains 13 to 30 or more nucleotides.

The nucleic acid may be isolated, extracted and/or purified from the sample prior to use in the method of the invention. The isolation, extraction and/or purification may be performed by any suitable technique. For example, the nucleic acid isolation, extraction and/or purification may be performed using a nucleic acid isolation kit, a nucleic acid extraction kit or a nucleic acid purification kit, respectively.

The method of the present disclosure may further comprise an initial step of isolating, extracting and/or purifying the nucleic acid from the sample. The method may therefore further comprise isolating the nucleic acid from the sample. The method may further comprise extracting the nucleic acid from the sample. The method may further comprise purifying the nucleic acid from the sample. Alternatively, the method may comprise direct amplification from the sample without an initial step of isolating, extracting and/or purifying the nucleic acid from the sample. Accordingly, the method may comprise lysing cells in the sample or amplifying free circulating DNA.

Following isolation, extraction and/or purification, the nucleic acid may be used immediately or may be stored under suitable conditions prior to use. Accordingly, the method of the invention may further comprise a step of storing the nucleic acid after the extracting step and before the amplifying step.

The step of obtaining the sample and/or the step of isolating, extracting and/or purifying the nucleic acid from the sample may occur in a different location to the subsequent steps of the method. Accordingly, the method may further comprise a step of transporting the sample and/or transporting the nucleic acid.

The method may further comprise diagnosing a pathogen, an infectious disease, antimicrobial resistance or a drug resistant infection if the nucleic acid molecule is present.

The infectious disease may be selected from the group consisting of Adenovirus, Coronavirus, Human Rhinovirus, Human Metapneumovirus, Parainfluenza, Respiratory Syncytial Virus, Bordetella Acute Flaccid Myelitis (AFM), Anaplasmosis, Anthrax, Babesiosis, Botulism, Brucellosis, Burkholderia mallei (Glanders), Burkholderia pseudomallei (Melioidosis), Campylobacteriosis (Campylobacter), Carbapenem-resistant Infection (CRE/CRPA), Chancroid, Chikungunya Virus Infection (Chikungunya), Chlamydia, Ciguatera, Clostridium Difficile Infection, Clostridium Perfringens (Epsilon Toxin), Coccidioidomycosis fungal infection (Valley fever), Creutzfeldt-Jacob Disease, transmissible spongiform encephalopathy (CJD), Cryptosporidiosis (Crypto), Cyclosporiasis, Dengue, 1,2,3,4 (Dengue Fever), Diphtheria, E. coli infection (E. Coli), Eastern Equine Encephalitis (EEE), Ebola, Hemorrhagic Fever (Ebola), Ehrlichiosis, Encephalitis, Arboviral or parainfectious, Enterovirus Infection, Non-Polio (Non-Polio Enterovirus), Enterovirus Infection, D68 (EV-D68), Giardiasis (Giardia), Gonococcal Infection (Gonorrhea), Granuloma inguinale, Haemophilus Influenza disease, Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster, zoster VZV (Shingles), Histoplasmosis infection (Histoplasmosis), Human Immunodeficiency Virus/AIDS (HIV/AIDS), Human Papillomarivus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LVG), Malaria, Measles, Meningitis, Viral (Meningitis, viral), Meningococcal Disease, Bacterial (Meningitis, bacterial), Middle East Respiratory Syndrome Coronavirus (MERS-COV), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis, Pthiriasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Including congenital (German Measles), Salmonellosis gastroenteritis (Salmonella), Scabies Infestation (Scabies), Scombroid, Severe Acute Respiratory Syndrome (SARS), Shigellosis gastroenteritis (Shigella), Smallpox, Staphyloccal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus Infection, tetani (Lock Jaw), Trichonosis Infection (Trichinosis), Tuberculosis (TB), Tuberculosis (Latent) (LTBI), Tularemia (Rabbit fever), Typhoid Fever, Group D, Typhus, Vaginosis, bacterial (Yeast Infection), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), Zika Virus Infection (Zika) and COVID-19.

The skilled person will be familiar with many amplification chemistries, and this disclosure is not limited to any particular chemistry or reaction. Similarly, the disclosure is not limited to any particular amplification instrument. Suitable amplification instruments include any instrument capable of real-time measurements including bulk (such as qPCR platform) or single-molecule (such as dPCR platform). The method can be used with single-channel or multi-channel instruments. For example, an instrument with 5 channels (i.e. each channel reads a different colour), may be used, in which 3 targets are multiplexed per channel, totaling 15 targets in a single reaction. Similarly, the present disclosure is not limited to any particular sensing method. Sensing methods may be (i) Fluorescent based, including probe-based (e.g. Taqman, Scorpion, FRET) or dye-based (e.g. SYBR. EvaGreen, SYTO). (ii) Colorimetric based. (iii) Electrochemical based (e.g. pH or ion based sensing).

For example, the nucleic acid amplification method may comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR, intersequence-specific PCR (ISS-PCR), whole genome amplification (WGA), inverse PCR, or thermal asymmetric interlaced PCR (TAIL-PCR).

In some embodiments, the nucleic acid amplification reaction may be a nucleic acid isothermal amplification method. Isothermal amplification is a form of nucleic acid amplification which does not rely on the thermal denaturation of the target nucleic acid during the amplification reaction and hence does not require multiple rapid changes in temperature. Isothermal nucleic acid amplification methods can therefore be carried out inside or outside of a laboratory environment. A number of isothermal nucleic acid amplification methods have been developed, including but not limited to Strand Displacement Amplification (SDA), Transcription Mediated Amplification (TMA), Nucleic Acid Sequence Based Amplification (NASBA), Recombinase Polymerase Amplification (RPA), Rolling Circle Amplification (RCA), Ramification Amplification (RAM), Helicase-Dependent Isothermal DNA Amplification (HDA), Circular Helicase-Dependent Amplification (cHDA), Loop-Mediated Isothermal Amplification (LAMP), Single Primer Isothermal Amplification (SPIA), Signal Mediated Amplification of RNA Technology (SMART), Self-Sustained Sequence Replication (3SR), Genome Exponential Amplification Reaction (GEAR) and Isothermal Multiple Displacement Amplification (IMDA). Further examples of such amplification chemistries are described in, for example, “Isothermal nucleic acid amplification technologies for point-of-care diagnostics: a critical review” (Pascal Craw and Wamadeva Balachandrana Lab Chip, 2012, 12, 2469-2486, DOI: 10.1039/C2LC40100B).

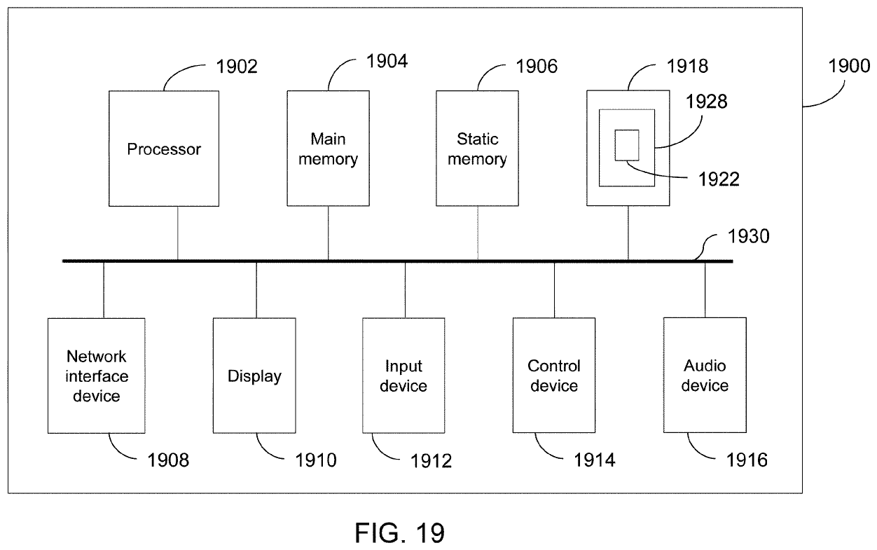

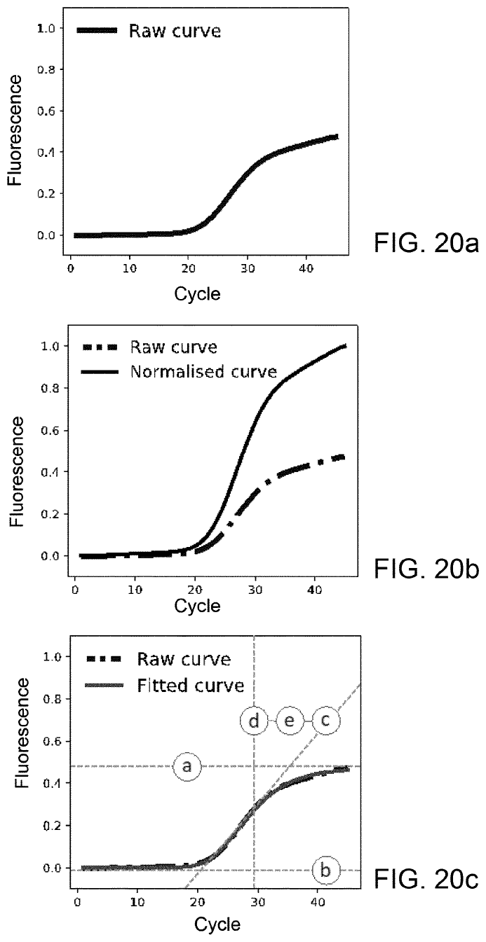

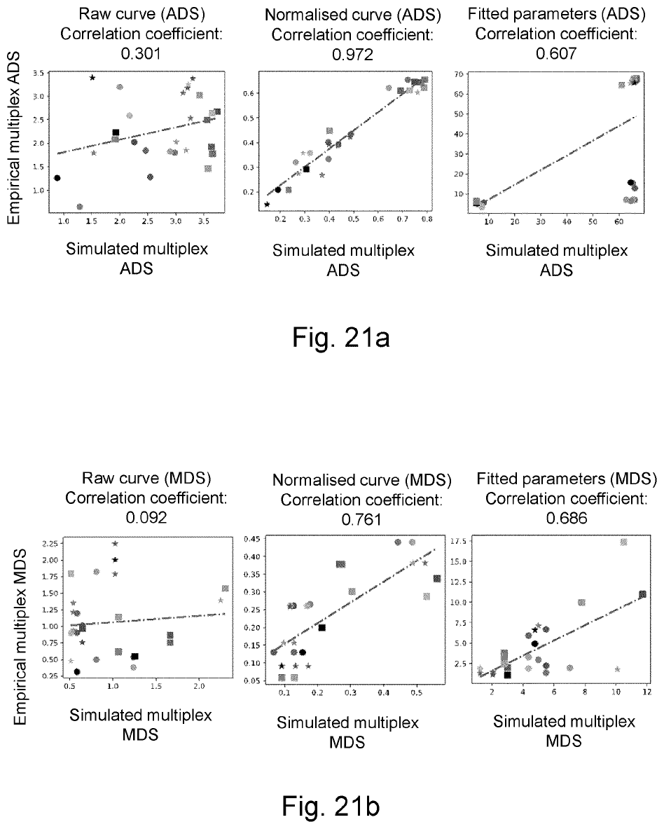

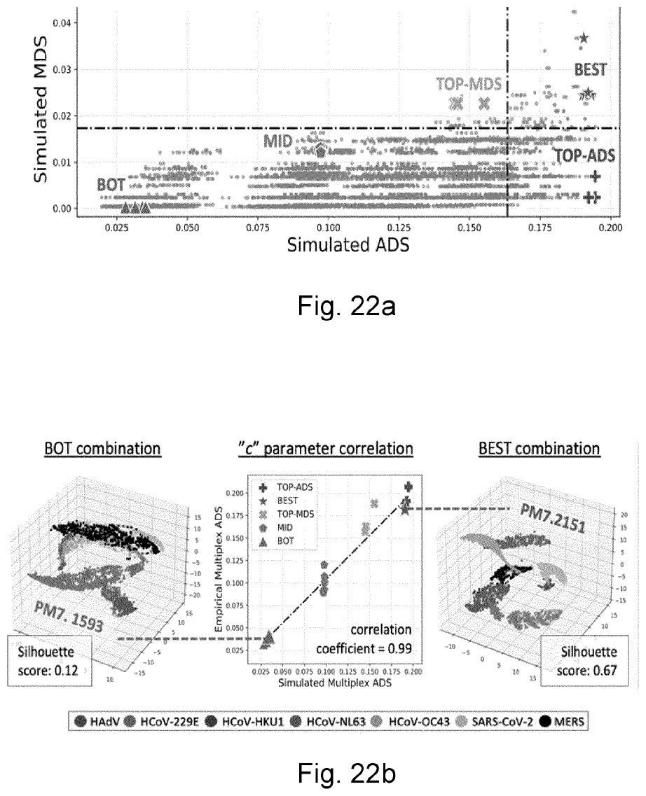

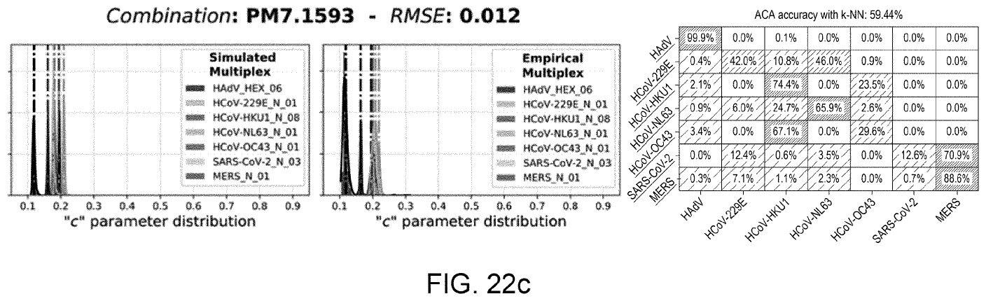

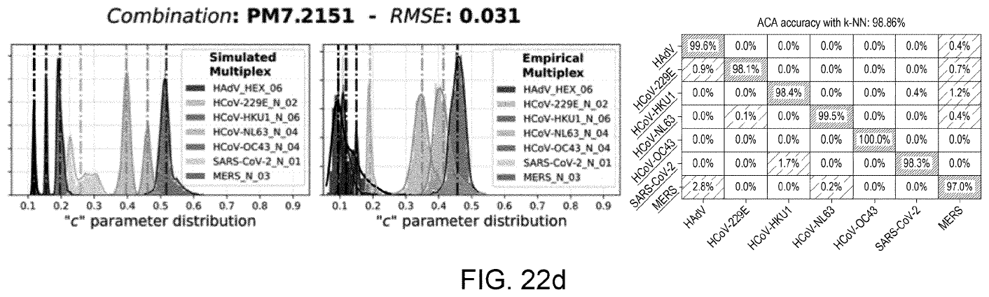

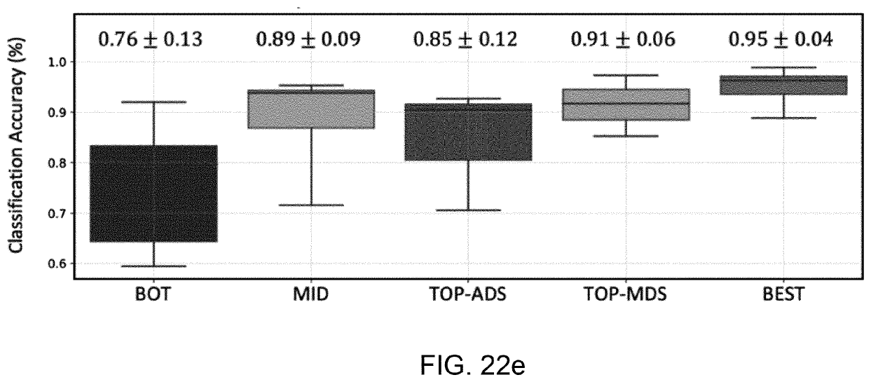

### A Computing Device and a Computer Readable Medium-FIG. 19

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

FIG. 19 illustrates a block diagram of one implementation of a computing device 1900 within which a set of instructions, for causing the computing device to perform any one or more of the methodologies discussed herein, may be executed. In alternative implementations, the computing device may be connected (e.g., networked) to other machines in a Local Area Network (LAN), an intranet, an extranet, or the Internet. The computing device may operate in the capacity of a server or a client machine in a client-server network environment, or as a peer machine in a peer-to-peer (or distributed) network environment. The computing device may be a personal computer (PC), an integrated circuit, a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single computing device is illustrated, the term “computing device” shall also be taken to include any collection of machines (e.g., computers) that individually or jointly execute a set (or multiple sets) of instructions to perform any one or more of the methodologies discussed herein.

The example computing device 1900 includes a processing device 1902, a main memory 1904 (e.g., read-only memory (ROM), flash memory, dynamic random access memory (DRAM) such as synchronous DRAM (SDRAM) or Rambus DRAM (RDRAM), etc.), a static memory 1906 (e.g., flash memory, static random access memory (SRAM), etc.), and a secondary memory (e.g., a data storage device 1918), which communicate with each other via a bus 1930.

Processing device 1902 represents one or more general-purpose processors such as a microprocessor, central processing unit, or the like. More particularly, the processing device 1902 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1902 may also be one or more special-purpose processing devices such as an application specific integrated circuit (ASIC), a field programmable gate array (FPGA), a digital signal processor (DSP), network processor, or the like. Processing device 1902 is configured to execute the processing logic (instructions 1922) for performing the operations and steps discussed herein.

The computing device 1900 may further include a network interface device 1908. The computing device 1900 also may include a video display unit 1910 (e.g., a liquid crystal display (LCD) or a cathode ray tube (CRT)), an alphanumeric input device 1912 (e.g., a keyboard or touchscreen), a cursor control device 1914 (e.g., a mouse or touchscreen), and an audio device 1916 (e.g., a speaker).

The data storage device 1918 may include one or more machine-readable storage media (or more specifically one or more non-transitory computer-readable storage media) 1928 on which is stored one or more sets of instructions 1922 embodying any one or more of the methodologies or functions described herein. The instructions 1922 may also reside, completely or at least partially, within the main memory 1904 and/or within the processing device 1902 during execution thereof by the computer system 1900, the main memory 1904 and the processing device 1902 also constituting computer-readable storage media.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM, CD-R/W or DVD.

In an implementation, the modules, components and other features described herein can be implemented as discrete components or integrated in the functionality of hardware components such as ASICS, FPGAs, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It will be understood that the above description of specific embodiments is by way of example only and is not intended to limit the scope of the present disclosure. Many modifications of the described embodiments, some of which are now described, are envisaged and intended to be within the scope of the present disclosure.

In one implementation, the method of FIG. 5 may optionally involve filtering the amplification data generated from a plurality of preparatory assays prior to determining a plurality of similarity metrics (block 530). Filtering may involve subtracting the curve background to remove fluorescence signal noise at the starting cycles. It may further involve removal of late amplification curves to exclude non-plateau reactions. It may further involve removal of noisy curves to exclude non-sigmoidal shapes, for example those that may result from operator or instrumentation faults.

Optionally, filtering may include applying an Adaptive Mapping Filter (AMF) to consider the variability of positive counts in digital PCR. Abnormalities may be linked to shifted melting distribution or decreased PCR efficiency. Classification accuracies may be compared before and after the AMR is applied, showing an improved sensitivity of 1.18% for inliers and 20% for outliers (p-value <0.0001).

The filtering framework is an intelligent algorithm that allows outliers to be filtered out from amplification events. It is capable of capturing kinetic and thermodynamic abnormalities of amplification curves. This results in more separated ACA clusters and clearer boundaries such that optimal primer sets can be more easily identified. AMF may involve calculating a hyperparameter called contamination ratio or an outlier percentage.

The input may be raw amplification curve data. Baseline and flat/late curve removal may be applied to this input. Then, each processed curve may be fitted by a sigmoid function. The fitting parameters may be used as input for the filtering algorithm which identifies outliers. The framework may output the filtered amplification curves, marked as inliers.

Optionally, the end slope (Send) is a feature that aims to provide further information about the amplification curve shape. It may be calculated by taking the average of the first derivatives at the last five cycles of the amplification curve:

$${S}_{e n d} = \frac{1}{5} [ D ( N - 4 ) D ( N - 3 ) … D ( N ) ] {e}_{5}^{T} where D ( x ) = \frac{d F ( t )}{d t} and {e}_{5} = [ 11111 ]$$

and N is the total cycle number. This feature can be used in addition to the fitting parameters to extract information about the amplification curve. In particular, this feature is used to extract information in the tail of the curve, which contributes to distinguishing inliers and outliers.

Alternative algorithms may be used to filter the amplification data including but not limited to proximity-based outlier detection algorithms (for example, using Euclidean or Manhattan distance metrics), outlier ensembles, and angle-based algorithms. Examples of proximity-based algorithms are Local Outlier Factor (LOF) and Density-based Spatial Clustering of Applications with Noise (DBSCAN). Examples of outlier ensembles are Isolation Forest and feature bagging.

The plurality of similarity metrics determined at block 530 in FIG. 5 are indicative of a degree of similarity between the amplification data produced by one of the plurality of preparatory assays compared to another one of the preparatory assays. In one implementation, the similarity may be determined using the entirety of the amplification data to determine the degree of similarity. For example, an amplification curve may be a time series where fluorescence values change as the number of cycles increases. This may be generated from a real-time PCR reaction. The term “raw” curve here refers to the raw amplification data. FIG. 20a depicts an example of a raw amplification curve after data processing.

In another implementation, the similarity may be determined at block 530 using normalized curves. This normalization may be performed using the final fluorescence intensity (FFI) as input to remove the absolute fluorescence information. FIG. 20b depicts an example of a normalized curve computed based on the Final Fluorescence Intensity (FFI) shown by an unbroken line, compared to a raw amplification curve in a dashed line.

In another implementation, the similarity may be determined at block 530 using sigmoidal parameters generated from a fitting model, for example a 5-parameter fitting model. In some implementations, this fitting model may be the same fitting model used to filter the amplification data.

Alternatively, 4-parameter and 6-parameter models may be used to model the real-time PCR sigmoid. An example of a 5-parameter sigmoid function is:

$$f ( t ) = \frac{a}{{( 1 + {exp}^{- c ( t - d )} )}^{e}} + b$$

where t is the amplification time (or PCR cycle), f(t) is the fluorescence at time t, a is the maximum fluorescence, b is the baseline of the sigmoid, c is related to the slope of the curve, d is the fractional cycle of the inflection point, and e allows for an asymmetric shape (Richard's coefficient).

FIG. 20c depicts an example of a fitted curve shown by an unbroken line, compared to a raw amplification curve in a dashed line.

The fitted curve (such as the example shown on the right graph of FIG. 20c) may be computed using a 5-parameter Sigmoid function where the input is the raw amplification curve. Fitted parameters (“a”, “b”, “c”, “d”, “e”) and a fitted curve can be obtained using this method. The fitted curve contains predicted fluorescence values corresponding to each cycle from the 5-parameter Sigmoid model with fitted parameters.

Determining the plurality of similarity metrics may comprise computing a distance measure. This measure may also be used to measure transferability from simulated to empirical multiplexes, and the transferability demonstrates that distances between amplification curves are maintained during the transition from singleplex to multiplex environments.

In a single channel multiplex assay, the number of primer sets present in the reaction equals the number of targets (Nₜ). Therefore, the number of distances (N_{d}) among curves of different targets is represented by the following formula:

$${N}_{d} = ( \frac{{N}_{t}}{2} ) = \frac{{N}_{t} ( {N}_{t} - 1 )}{2}$$

A first distance metric which may be used to determine a similarity metric is average distance score (ADS). This provides information on the overall distances across targets. The higher its values are, the more distant the curves are, and therefore a better ACA performance is expected as distances are related to data point clusters.

For example, this method may be evaluated by designing three primer sets for three selected targets using synthetic DNA and testing them in real-time digital PCT (qdPCR): Adenovirus (HAdV), Human coronavirus HKU1 (HCoV-HKU1) and Middle East respiratory syndrome-related coronavirus (MERS-COV). The number of combinations to test using Nₜ targets (Nₜ=3) and N_{Ps} assays for each target (N_{Ps}=3) is 27 (N_{c}=N_{Ps}^{N}^{ₜ}=27) combinations. A complete comparison of all the 27 simulated and empirical multiplex assays can be conducted, since the number of wet-lab experiments is achievable ((N_{c}×Nₜ=81 tests).

FIG. 21a shows the correlations of the ADS between simulated and empirical multiplexes for three types of curves or parameters for the 27 combinations for a 3-plex assay. From left to right, these three types are raw curve, normalized curve and fitted parameters. Each point with a unique shape corresponds to combination 1 to 27. The dashed lines are computed using linear regression. The Pearson coefficients for all three plots are calculated, and are 0.301 for the raw curve, 0.972 for the normalized curve, and 0.607 for the fitted curve.

A first distance metric which may be used to determine a similarity metric is minimum distance score (MDS). A high ADS does not necessarily mean that there will be a large distance between every two targets of the multiplex, for example, there may be extreme outliers that skew the score. MDS may be used alternatively or additionally to MDS to provide the distance value of the two closest curves or the minimum value of the given Nu distances.

FIG. 21b shows the correlations of the MDS between simulated and empirical multiplexes for three types of curves or parameters for the 27 combinations. From left to right, these three types are raw curve, normalized curve and fitted parameters. Each point with a unique shape corresponds to combination 1 to 27. The dashed lines are computed using linear regression. The Pearson coefficients for all three plots are calculated, and are 0.092 for the raw curve, 0.761 for the normalized curve, and 0.686 for the fitted curve.

In a preferred implementation, the similarity metrics may depend on both average and minimum distance scores. A viability score may be assigned to each of the plurality of trial multiplex assays based on these scores.

Distances among amplification curves of empirical multiplex assays are similar to those generated in simulated multiplexes. Therefore, leveraging ADS and MDS for simulated multiplexes can be used to rank each combination and find the optimal assays with the largest inter-target distances for the ACA classifier.

The ADS and MDS may be used to narrow down the selection of empirical testing for the highest performing multiplexes using a ranking system. They can be also be used to validate that inter-curve distance information is maintained during the transition from simulated to empirical multiplexes, and so they can be used to develop assays in silico that are more suitable for ACA. This results in a reduced resource cost, as it reduces expensive and time-consuming laboratory testing.

As discussed in previous implementations, determining the plurality of similarity metrics may comprise computing a distance measure between the data distributions of the one of the plurality of preparatory assays and the another one of the plurality of preparatory assays. Determining the plurality of similarity metrics may further comprise calculating an average distance score for each combination of targets and primer sets used in the preparatory assays, and calculating a minimum distance score for each combination of targets and primer sets used in the preparatory assays.

The data distribution may comprise normalized amplification data. Most preferably, normalized curves may be used to determine ADS and MDS. In FIGS. 21a and 21b, both ADS and MDS showed the maximum correlation values when considering normalized curves (the center graphs of FIGS. 21a and 21b). Reducing the information contained in the amplification curve is beneficial. When computing a distance measure between the data distributions, the data distributions may comprise normalized amplification data.

In a 3-plex validation, each singleplex assay can be tested against its specific target (N=9), resulting in 27 different combinations of simulated multiplexes. In one implementation, the plurality of similarity metrics are computing using data fitted using the “c” parameter. In one example, the “c” parameter can be fitted and extracted from 27 empirically tested multiplex assays (corresponding to 81 tests). The “c” parameter distribution is maintained when translated to empirical multiplexes. In other words, the “c” parameter is capable of maintaining distance information going from simulation to empirical test.

When computing a distance measure between the data distributions, the data distributions may comprise at least one fitted parameter. In a preferred implementation, the at least one fitted parameter is the extracted “c” parameters.

In most cases, the location of the parameter distribution for each target is maintained when going from simulation to empirical test. In other situations, the distribution may be shifted from the singleplex events, while the relative distance relationship of “c” values is maintained. For example, a low-rank ADS/MDS multiplex may show overlaps in the “c” parameter distribution for singleplex assays in both simulated and empirical multiplexes. As distances among amplification curve shapes can significantly affect the ACA classifier, reduced performance may be expected for multi-target identification.

Another distribution trend among multiplex assays may occur when there is high simulated ADS value, but low MDS. Therefore, considering minimum distance between “c” parameter distributions of the two closest targets may be used. A small MDS value indicates a less separable group of target clusters, resulting in low ACA accuracies for multi-pathogen identification in a single fluorescent channel reaction.

The data distribution may comprise at least one fitted parameter of the amplification data. In one preferred implementation, ADS and MDS may be computed from the “c” parameter of the data.

The inter-target curve shape differences may be increased using various other methods, not limited to the methods described above. For example, probe-based chemistries may be used to modify amplification curve shapes by changing the concentration levels of the fluorescent prove in order to enlarge inter-target distances and ease the ACA classification with better clustering performance. These methods may be used individually or in combination with one another.

FIG. 22 depicts validation of a method based on 7-plex assays.

In another example, the method of FIG. 5 can be used to identify an optimal 7-plex assay which, through the ACA method, is able to accurately identify the following Respiratory Tract Infection (RTI) pathogens in a single fluorescent channel using qdPCR: Human adenovirus (HAdV), Human coronavirus OC43 (HCoV-OC43), Human coronavirus HKU1 (HCoV-HKU1), Human coronavirus 229E (HCoV-229E), Human coronavirus NL63 (HCoV-NL63), Middle East respiratory syndrome-related coronavirus (MERS-COV), and Severe acute respiratory syndrome coronavirus 2 (SARS-COV-2). There are at least two different assays for each target, for a total of 24 singleplexes across the seven pathogens. All possible 7-plex combinations (N=4608) can be analysed, and their ADS and MDS calculated in order to determine a similarity metric for each combination and determine the optimal primer sets.

FIG. 22a depicts 2-D ranking results for all 4608 combinations in 7-plex based on simulated ADS and simulated MDS. This figure shows how the ADS and MDS can be visualized in a two-dimensional space. By considering the mean and standard deviation of the two scores, we set up boundaries to the ADS/MDS distribution for all the combinations and divided the space into four separate regions, demonstrating how empirical multiplexes would perform for the ACA method depending on their ADS/MDS. The black horizontal segmented line in FIG. 5a divides high and low MDS, and the vertical segmented line separates the high and low ADS regions, resulting in four distinct areas. Empirical testing of different multiplexes from each of these regions demonstrates that the chance of developing a reliable multiplex can vary based on the selected regions or selection criteria. Therefore, multiplex assays can be selected from different areas and categorized into five classes: BOT (N=6), MID (N=6), BEST (N=6), TOP-ADS and TOP-MDS (N=6) values. These five classes can then be empirically tested with synthetic DNA in qdPCR.

FIG. 22b depicts the distances of the “c” parameters of each selected multiplex compared to the simulated one. The 2-D plot in the middle of FIG. 22b depicts the relationship between empirical and simulated scores based on “c” parameters, with a correlation coefficient of 0.99. Enlarged data points for one of the BOT (PM7.1593) and BEST (PM7.2151) combinations are visualized with 3-D t-SNE on raw curves, and the corresponding Silhouette scores are calculated. The Silhouette score for the BOT combination is 0.12, and 0.67 for the BEST combination.

FIG. 22c depicts simulated and empirical “c” distributions of the selected combination BOT (PM7.1593). FIG. 22d depicts simulated and empirical “c” distributions of the selected combination BEST (PM7.2151). The vertical dashed lines correspond to the mean of the distribution computed for different targets. On the right, the confusion matrixes of ACA performance for both cases are presented, and overall accuracy using k-NN is reported in the title. True labels are on the y-axis and ACA predicted labels are on the x-axis (each target sensitivity is also reported in percentage). These figures show a small RMSE for both BOT and BEST assays (0.012 and 0.031), and confirm the distance-maintaining hypothesis validated in the 3-plex experiments. Moreover, the ACA accuracy is validated using training and testing datasets obtained in different experimental settings (different days, operators, and reagents) to ensure the reproducibility of the methodology. As expected, the performance of the BEST combination was significantly higher than the BOT one, with a 39.42% increase in accuracy.

FIG. 22e depicts a box plot of ACA classification accuracy for each selected group. The mean and standard deviation of ACA accuracy on empirical multiplexes are calculated and shown on each box bar. The BEST combination group scored an average (±standard deviation) classification performance of 95% (±0.04%) using a k-NN classifier, which is the highest average and the lowest standard deviation among all the groups. There is a decreasing trend in the average accuracy, and an increasing trend in the standard deviation as the ADS/MDS values become smaller. Previously, the 3-plex validation showed the presence of outliers in low ADS/MDS rank with high ACA classification accuracy, which is also observed in these 7-plex tests.

It is therefore possible to select the highest rank combination in silico with wet-lab tested singleplexes, avoiding performing expensive and time-consuming multiplex assay development phases. This method represents a solution for developing multiplex assays by utilising both empirical testing and in-silico computation.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A computer-implemented method for determining optimal primer sets for a multiplex assay, each of the optimal primer sets intended to amplify one or more targets, the method comprising:
obtaining amplification data from a plurality of preparatory assays, the amplification data describing at least:
the amplification of a first target of the one or more targets by a first primer set in a first preparatory assay,
the amplification of the first target amplified by a second primer set in a second preparatory assay,
the amplification of a second target of the one or more targets by the first primer set in a third preparatory assay, and
the amplification of the second target by the second primer set in a fourth preparatory assay;
the method further comprising:
determining a plurality of similarity metrics, each similarity metric being indicative of a degree of similarity between the amplification data produced by one of the plurality of preparatory assays compared to another one of the preparatory assays; and
determining, based on the plurality of similarity metrics, the optimal primer sets for the multiplex assay.

2. The computer-implemented method of claim 1, wherein a similarity metric is determined for each possible pairing of the preparatory assays.

3. The computer-implemented method of claim 1, further comprising determining a viability score for each of a plurality of trial multiplex assays, the trial multiplex assays comprising trial primer sets, and the viability score being based on similarity metrics associated with each of the trial primer sets;
wherein determining the optimal primer sets based on the plurality of similarity metrics comprises selecting the optimal primer sets from among the trial primer sets based on the ranking of the viability scores.

4. The method of claim 1, wherein determining the optimal primer sets further comprises:
constructing a similarity matrix of similarity metrics, the similarity matrix representing every combination of target and primer set used in the preparatory assays;
constructing sub-matrices from the similarity matrix, wherein each sub-matrix is indicative of a trial multiplex assay comprising trial primer sets, and the sub-matrix values are the similarity metrics associated with the trial primer sets; and
assigning a viability score to each trial multiplex assay based on the similarity scores within each submatrix.

5. The method of claim 4, wherein determining the optimal primer sets based on the plurality of similarity metrics comprises selecting the optimal primer sets from among the trial primer sets based on the viability scores.

6. The method of claim 4, further comprising applying constraints to each sub-matrix of preparatory assays, prior to determining a viability score.

7. The method of claim 1, wherein determining the plurality of similarity metrics comprises computing a distance measure between the data distributions of the one of the plurality of preparatory assays and the another one of the plurality of preparatory assays.

8. The method of claim 7, wherein the distance measure is one of Euclidean distance, Mahalonbis distance, Pearson Correlation, or Wasserstein distance.

9. (canceled)

10. The method of claim 7, wherein determining the plurality of similarity metrics further comprises:
calculating an average distance score for each combination of targets and primer sets used in the preparatory assays;
calculating a minimum distance score for each combination of targets and primer sets used in the preparatory assays.

11. (canceled)

12. The method of claim 7, wherein the data distribution comprises at least one fitted parameter of the amplification data.

13. The method of claim 4, wherein assigning the viability scores to each trial multiplex assay is based on a sum of the distances between the sub-matrix values.

14. The method of claim 4, wherein assigning the viability scores to each trial multiplex assay is based on a minimum distance between any two of sub-matrix values.

15. The method of claim 13, wherein assigning the viability scores to each trial multiplex assay is based on the product of the sum of the distances and the minimum distance.

16. The method of claim 1, wherein the amplification data is at least one of:
melting curve data;
amplification curve data;
fluorescence intensity data; or
non-fluorescence data such as electrochemical, colorimetric or pH-based signal data.

17. The method of claim 1, wherein the preparatory assays are singleplex assays.

18. The method of claim 1, wherein at least some of the plurality of preparatory assays are low-level multiplex assays, and the multiplex assay is a higher-level multiplex assay.

19. (canceled)

20. The method of claim 1, wherein the amplification data describes the amplification of a plurality of different combinations of targets by a plurality of different primers or primer sets.

21. The method of claim 1, wherein the multiplex assay is intended to identify a plurality of identifiable targets, and the optimal primer sets are intended to enable amplification of each of those identifiable targets to produce amplification data from which the amplification activity of each identifiable target can be distinguished from the amplification activity of every other identifiable target.

22. A computer readable medium comprising computer executable instructions which, when performed by a processor, cause the processor to perform a method comprising:
obtaining amplification data from a plurality of preparatory assays, the amplification data describing at least:
the amplification of a first target of the one or more targets by a first primer set in a first preparatory assay,
the amplification of the first target amplified by a second primer set in a second preparatory assay,
the amplification of a second target of the one or more targets by the first primer set in a third preparatory assay, and
the amplification of the second target by the second primer set in a fourth preparatory assay;
the method further comprising:
determining a plurality of similarity metrics, each similarity metric being indicative of a degree of similarity between the amplification data produced by one of the plurality of preparatory assays compared to another one of the preparatory assays; and
determining, based on the plurality of similarity metrics, the optimal primer sets for the multiplex assay.

23. A system comprising:
one or more processors; and
a computer-readable medium including one or more instructions that, when executed by one or more processors, cause the system to perform a method comprising:
obtaining amplification data from a plurality of preparatory assays, the amplification data describing at least:
the amplification of a first target of the one or more targets by a first primer set in a first preparatory assay,
the amplification of the first target amplified by a second primer set in a second preparatory assay,
the amplification of a second target of the one or more targets by the first primer set in a third preparatory assay, and
the amplification of the second target by the second primer set in a fourth preparatory assay;
the method further comprising:
determining a plurality of similarity metrics, each similarity metric being indicative of a degree of similarity between the amplification data produced by one of the plurality of preparatory assays compared to another one of the preparatory assays; and
determining, based on the plurality of similarity metrics, the optimal primer sets for the multiplex assay.
